(19) United States
(12) Patent Application Publication
(10) Pub. No.: US 2020/0370138 A1
(43) Pub. Date: Nov. 26, 2020

(54) Amorphous Sugar Composition

(71) Applicant: Nutrition Science Design Pte. Ltd, Bedok Gardens (SG)

(72) Inventors: David Kannar, Bedok Gardens (SG); Meng Wai Woo, Bedok Gardens (SG); Yongmei Sun, Bedok Gardens (SG)

(21) Appl. No.: 16/966,305

(22) PCT Filed: Jan. 31, 2019
(86) PCT No.: PCT/SG2019/050057

§ 371 Date: Jul. 30, 2020

(30) Foreign Application Priority Data
Jan. 31, 2018 (SG) 10201800837U
Oct. 18, 2018 (SG) 10201809207W

(51) Int. Cl. C13B 50/00; A23L 33/105; A23L 33/125; A23P 10/43; A23L 33/21; A21D 2/18; A23G 1/40

(52) U.S. Cl. CPC C13B 50/002; A23L 33/105; A23L 33/125; A23V 2002/00; A23L 33/21; A21D 2/181; A23G 1/40; A23P 10/43

(57) ABSTRACT

The present invention provides an amorphous sugar comprising sucrose, at least about 20 mg CE polyphenols/100 g carbohydrate and a drying agent with a low glycaemic index. The invention further provides an amorphous sugar comprising one or more sugars and a drying agent with a low glycaemic index. The amorphous sugar of the invention may optionally further comprise prebiotics, alternative sweeteners, proteins and lipids. The amorphous sugar of the invention may optionally be aerated. The invention further provides methods of making the amorphous sugar including by rapidly drying, such as spray drying. The invention further provides methods of preparing aerated amorphous sugar. The invention further provides methods of food and beverage preparation using the amorphous sugar.

### FIELD OF THE INVENTION

The present invention relates to sugar compositions, sugar derived compositions and processes for the preparation of said compositions. The present invention further relates to compositions comprising alternative sweeteners and processes for the preparation of said compositions. In some embodiments, the present invention relates to sugar compositions, sugar derived compositions and alternative sweetener compositions with a low glycaemic response (GR), low glycaemic index (GI) and/or low glycaemic load (GL) and processes for their preparation. In some embodiments, the present invention relates to sugar compositions, sugar derived compositions and alternative sweetener compositions having reduced calorific content and/or lowered bulk density and processes for their preparation. The present invention further relates to foods and beverages containing and/or prepared using the sugar, sugar derived and/or alternative sweetener compositions of the invention, preferably the sugar and beverages have a reduced sugar content.

### BACKGROUND OF THE INVENTION

There is concern that refined white sugar is causal in the development of diabetes and obesity. Consequently, there is demand for alternatives to white refined sugar products, especially if the product is likely to provide health benefits or minimise the health risks.

Many efforts have been made to replace or reduce white sugar using artificial sweeteners and/or honey. However, the use of some artificial sweeteners has now also been directly correlated with increased risks of type II diabetes as well as the acceleration of obesity and inhibition of fat break down. Artificial sweeteners may also change gut microflora and products formulated with these products may need to contain laxative warnings. Honey bee populations are also in decline limiting the quantity of honey available for use as a large-scale sugar substitute.

Current sugars include refined white sugar, brown sugar and “raw sugar”. All of these are crystalline sugars. The refining process used to prepare refined white sugar removes most vitamins, minerals and phytochemical compounds from the sugar leaving a “hollow nutrient”, that is, a food without significant nutritional value beyond the energetic value of the sugar.

Retention of vitamins, minerals and phytochemicals in sugar has been demonstrated to improve health and lower glycaemic index (GI) in some circumstances (see Jaffé, W. R., Sugar Tech (2012) 14:87-94). This is useful because it is thought that individuals who are susceptible to type II diabetes, obesity and coronary heart disease should follow a low GI diet. It is also recommended for these individuals to reduce sugar consumption. It has also been found that following a low GI and/or low calorie diet can assist individuals with diabetes to manage their sugar levels and also assist individuals with obesity problems to control food cravings, reduce appetite swings and improve eating habits.

Glycaemic response (GR) refers to the changes in blood glucose after consuming a carbohydrate-containing food. The glycaemic index is a measure of GR. It is a system for classifying carbohydrate-containing foods that generally correlates with how fast they raise blood-glucose levels inside the body. Low GI foods cause slow rises in blood-sugar. High GI foods trigger strong insulin responses. Frequently repeated strong insulin responses are thought to, over time, result in an increased risk of diabetes. Low GI foods do not trigger as high an insulin response.

Low GI crystalline sugars have been produced. However, the vast majority of the sugar used as an ingredient in industry is still refined white sugar. Therefore, there is still a need for additional low GI sugars in the food industry. There is also a need for low GI sugar that can be produced at lower cost and/or with low hygroscopicity so that it has a suitable shelf life and/or can be prepared in industrial quantities.

Low hygroscopicity is important because hygroscopicity makes the sugar difficult to use and store. This is particularly disadvantageous in an industrial setting because of the tendency for the sugar to clump and stick to equipment. Working with hygroscopic sugar in an industrial setting may require, for example, equipment operating under nitrogen to minimise the quantity of sugar that clumps or sticks to the equipment. Hygroscopic sugars can be sold in small retail products but they are not ideal for industrial use in the preparation of other foods, such as, chocolate, beverages, cereals, confectionary, bakery goods and other retail foods containing sugar.

Rapid drying, such as spray drying, is a technique used in food preparation, for example, to prepare milk powder. Unfortunately, it is difficult to spray dry sugar products because of the problems with stickiness and caking that occur when drying sugar-rich liquids containing high quantities of low molecular weight carbohydrates (LMWCs) such as sucrose, which have low glass transition temperatures, thus making the product sticky at ambient or high temperatures. Stickiness reduces the flowability and yield of powder whilst also causing equipment to clog. There can also be problems when the product is heated above its glass transition temperature during drying. One solution that is available is the addition of high molecular weight carbohydrates (HMWCs) to increase the glass transition temperature of the solution. Unfortunately, the HMWCs in use for food products, such as maltodextrin, have high GI.

There is a need for alternatives to traditional sugars. These alternatives can take the form of non-traditional sugars and/or alternative sweeteners to minimise the waste of sugar production, increase the efficiency of sugar processing and/or lessen the health risks associated with the consumption of sugar. It is useful if the non-traditional sugar or alternative sweetener is low GR, low GI and/or low GL. It is useful if the non-traditional sugar or alternative sweetener has reduced calories by weight or volume compared to traditional white sugar.

It is particularly useful if a non-traditional sugar or alternative sweetener is inexpensive to produce and suitable for use in commercial scale food production because, for example, it has suitably low hygroscopicity and/or fast dissolution.

There is also a need for sugar reduction strategies for foods and beverages to minimise the calories traditionally present in the food or beverage.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides an alternative to traditional crystalline sugar. The sugar of the present invention is largely amorphous. This is different to traditional sugars used in food preparation, which are crystalline because they are prepared by concentrating sugar cane or beet juice, crystallising the resulting syrup to form sugar crystals and removing the uncrystallised syrup (ie molasses). Instead, the amorphous sugar of the invention can be prepared by rapid drying, such as spray drying, a liquid containing sucrose and polyphenols, such as sugar juice or molasses or a combination thereof. The sucrose can be substituted for glucose or fructose etc. The polyphenols, which are present to lower the GI, are not necessary for effective preparation of the amorphous sugar and can be reduced or removed when a low GI sugar is not needed or the polyphenol GI lowering effect is not needed, for example, for a fructose sugar, which is inherently low GI.

Sucrose Sugars

Traditionally, molasses has been considered an unprofitable by-product of sugarcane processing, and has essentially only been used as an additive in feedstock for cattle and other animals. The use of spray dried molasses as an alternative sugar for human use would increase the sugar supply. The use of sugar juices such as sugar cane juice allows for preparation of a sugar product without the need to generate by-products like molasses. The single step process required for the preparation of rapidly dried sugar products is vastly more efficient that the preparation of traditional crystalline sugars. The preparation of this type of sugar also minimises the generation of waste products and retains nutrients in the sugar.

In a first aspect, the present invention provides an amorphous sugar comprising sucrose, at least about 20 mg catechin equivalent (CE) polyphenols/100 g carbohydrate and a low GI drying agent.

There are multiple options for the measurement of polyphenol content. One option is to measure milligrams catechin equivalents (CE) per amount of carbohydrate. An alternative is to measure gallic acid equivalents (GAE) per amount of carbohydrate. Amounts in mg CE/100 g can be converted to mg GAE/100 g by multiplying by 0.81 ie 60 mg CE/100 g is 49 mg GAE/100 g.

In an alternate first aspect, the present invention provides an amorphous sugar comprising sucrose, at least about 20 mg CE polyphenols/100 g carbohydrate and one or more edible, high molecular weight, low GI drying agents.

In an alternate first aspect, the present invention provides an amorphous sugar comprising sucrose, at least about 20 mg CE polyphenols/100 g carbohydrate and one or more edible, high molecular weight, low GI drying agents selected from the group consisting of lactose, protein, low GI carbohydrates, insoluble fibre, soluble fibre, lipids, natural intense sweeteners and/or combinations thereof.

In one embodiment, the present invention provides an amorphous sugar comprising 40% to 95% w/w sucrose, 0% to 4% w/w reducing sugars, at least about 20 mg CE polyphenols/100 g carbohydrate to about 1 g polyphenols CE/100 g carbohydrate and 5% to 60% w/w low GI drying agent selected from lactose, a low GI carbohydrate and/or a protein.

The low GI drying agent for the first and alternate first aspects of invention are described below as is the polyphenol content.

The amorphous sugar of the first or alternative first aspects of the invention optionally further comprises reducing sugars such as fructose and/or glucose.

Previous research indicates that sugar with the claimed amount of polyphenols will be low glycaemic so long as the quantity of higher GI sugars like glucose is low. If the drying agent is also low glycaemic or no glycaemic the amorphous sugar will also be low glycaemic. The amorphous sugar of the first or alternative first aspects of the invention is optionally low glycaemic and/or low glycaemic load.

An amorphous sugar according to the first or alternate first aspects of the invention can be prepared from either sugar cane or sugar beet or from refined white sugar (ie sucrose sugar sources). Beet sugar does not contain polyphenols and neither does refined white sugar contain more than trace amounts of polyphenols. However, polyphenols can be added to either to prepare a sugar according to the invention. The further polyphenols may be added to the sugar in a powdered or liquid form.

The amorphous sugar optionally has 40% to 95% w/w, 50% to 90% w/w or 50 to 80% w/w sucrose. Optionally, the reducing sugars are 0% to 4% w/w, 0.1% to 3.5% w/w, 0% to 3% w/w, 0% to 2.5% w/w, 0.1% to 2% w/w of the amorphous sugar. The amorphous sugar optionally has <0.3% w/w reducing sugars. This is of particular interest where the sucrose is sourced from sugar cane or sugar beet juice or molasses.

In some embodiments, the sucrose is sourced from cane juice, beet juice and/or molasses. In these embodiments, the drying agent is optionally whey protein isolate and/or sunflower protein.

Optionally, the sucrose is sourced from cane juice, beet juice and/or molasses and the drying agent is a digestive resistant carbohydrate.

Optionally, the sucrose is sourced from cane juice, beet juice and/or molasses and the drying agent is monk fruit.

Where the sucrose is sourced from beet juice the polyphenols will need to be measured. Cane juice and molasses may include sufficient polyphenols inherently, although additional polyphenols can be added if needed.

The amorphous sugar of the first and alternate first aspects of the invention optionally remains a free flowing powder following 6, 12 or 18 months storage in ambient conditions.

Low Molecular Weight Sugars

In a second aspect, the present invention provides an amorphous sugar comprising (i) one or more monosaccharides selected from the group consisting of glucose, fructose, galactose, ribose and xylose, and (ii) a low GI drying agent. Optionally the monosaccharide is glucose and/or fructose.

As described above, the low molecular weight sugar (including monosaccharides) have traditionally been difficult to prepare in amorphous form by rapid drying, such as spray drying. The development of the low GI drying agent has allowed preparation of dry, flowable amorphous powders from low molecular weight sugars such as monosaccharides while retaining a low GI.

In an alternative second aspect, the present invention provides an amorphous sugar comprising one or more low molecular weight sugars, at least about 20 mg CE polyphenols/100 g carbohydrate and a low GI drying agent.

In an alternate second aspect, the present invention provides an amorphous sugar comprising one or more low molecular weight sugars, at least about 20 mg CE polyphenols/100 g carbohydrate, and one or more edible, high molecular weight, low GI drying agents.

In an alternate second aspect, the present invention provides an amorphous sugar comprising one or more low molecular weight sugars, at least about 20 mg CE polyphenols/100 g carbohydrate and one or more edible, high molecular weight, low GI drying agents selected from the group consisting of lactose, protein, low GI carbohydrates, insoluble fibre, soluble fibre, lipids, natural intense sweeteners and/or combinations thereof.

The low molecular weight sugar in the alternate second aspects of the invention is optionally selected from the group consisting of sucrose, glucose, galactose, ribose, xylose, fructose and combinations thereof. The low molecular weight sugar in the alternate second aspects of the invention is optionally selected from the group consisting of sucrose, glucose, galactose, ribose, xylose and combinations thereof. The sugar is optionally sucrose, glucose and/or fructose. In some embodiments the low molecular weight sugar is sucrose and/or glucose.

A person skilled in the art would appreciate that inclusion of fructose could increase hygroscopicity and decrease shelf-life. Such products are best for prompt use rather than long term storage. Alternatively, their shelf life can be improved by low humidity storage among other options.

The amorphous sugar optionally has 40% to 95% w/w, 50% to 90% w/w or 50 to 80% w/w monosaccharide or low molecular weight sugar.

The amorphous sugar of the second and alternate second aspects of the invention optionally remains a free flowing powder following 6, 12 or 18 months storage in ambient conditions.

Options for the First and Second Aspects of Invention

In the first, second and their alternate aspects of the invention, it is preferred for the amorphous sugar to comprise relatively homogenous particles where each particle comprises both the drying agent and the sucrose/monosaccharide/low molecular weight sugar.

The amorphous sugar of the first and second aspects of invention and their alternatives optionally has a maximum of 1 g CE polyphenols/100 g carbohydrate. Without being bound by theory, the drying agent is thought to increase the overall glass transition temperature, allowing cane juice, molasses or a combination of the two to be dried without becoming sticky or caking. A similar effect is observed for pure sucrose (eg white refined sugar), glucose, fructose and other monosaccharides. As the drying agents traditionally used in spray drying are high GI, for example, maltodextrin, new drying agents have been utilised for this amorphous sugar. The newer substrates aim to reduce or maintain the reduction in the glycaemic index of the amorphous sugar and/or the glycaemic load of an amount of the amorphous sugar. In preferred embodiments, the amorphous sugar has a low GL and/or a low GI. Optionally, the amorphous sugar is food grade, that is, suitable for consumption.

One advantage of the use of an amorphous sugar is that an amorphous sugar will have faster dissolution than a crystalline sugar. Use of the amorphous sugar in the preparation of industrial food products would minimise the time taken to dissolve the sugar into, for example, a beverage.

Another advantage of the amorphous sugar is that higher amounts of polyphenols can be present than have been included in low GI crystalline sugars. In international patent application no PCT/AU2017/050782, a low GI crystalline sugar is described. The preparation of that crystalline sugar was based on the identification of a “sweet spot” in the level of sugar processing (ie the amount the massecuite is washed) where:
1. the reducing sugar content is low enough that the sugar is low hygroscopicity and the reducing sugars are not raising the GI of the sucrose; and
2. the polyphenol content remains high enough to lower the GI of the sucrose.

More specifically, the crystalline sugar included about 0 to 0.5 g/100 g reducing sugars and about 20 mg CE polyphenols/100 g carbohydrate to about 45 mg CE polyphenols/100 g carbohydrate and the sugar particles have a glucose based glycaemic index of less than 55. The amorphous sugar of this invention can contain much higher polyphenol content without the need to add extraneous polyphenols if the sugar source is sugar cane juice or molasses rather than the crystallised sugar and massecuite that remain after molasses is removed. Use of molasses as the sugar source also increases the caramel flavour of the sugar. While sugar beet juice can be used as a sugar source, it has no inherent polyphenols so those will need to be added to prepare a sugar according to the first, first alternative and second alternative aspects of invention.

Optionally, the amorphous sugar of the first, second or their alternative aspects of invention comprise about 20 mg CE polyphenols/100 g carbohydrate to about 1 g CE polyphenols/100 g carbohydrate, about 20 mg CE polyphenols/100 g carbohydrate to about 800 mg CE polyphenols/100 g carbohydrate, about 20 mg CE polyphenols/100 g carbohydrate to about 500 mg CE polyphenols/100 g carbohydrate, about 30 mg CE polyphenols/100 g carbohydrate to about 200 mg CE polyphenols/100 g carbohydrate, or about 20 mg CE polyphenols/100 g carbohydrate to about 100 mg CE polyphenols/100 g carbohydrate.

Alternatively, the amorphous sugar comprises about 50 mg CE polyphenols/100 g carbohydrate to about 100 mg CE polyphenols/100 g carbohydrate, 50 mg CE polyphenols/100 g carbohydrate to about 80 mg CE polyphenols/100 g carbohydrate, 50 mg CE polyphenols/100 g carbohydrate to about 70 mg CE polyphenols/100 g carbohydrate, 55 mg CE polyphenols/100 g carbohydrate to about 65 mg CE polyphenols/100 g carbohydrate. In some embodiments there is about 60 mg CE polyphenols/100 g carbohydrate. Preferably, the polyphenols are polyphenols that naturally occur in sugar cane (although they do not need to be sourced from sugar cane).

It is preferred that the polyphenols added to the sugar are polyphenols that, even if not sourced from sugar cane, are present in sugar cane. The polyphenols can be sourced from sugar cane, for example, from a sugar processing waste stream and may be in the form of a sugar cane extract.

Optionally, the amorphous sugar of the first and second aspects of invention and their alternatives has good or excellent flowability. Optionally, the amorphous sugar has 0 to 0.3% w/w moisture content. Alternatively, the amorphous sugar has 0 to 10% w/w moisture content, 0.1 to 8% w/w moisture content or 0.1 to 5% w/w moisture content.

Aerated versions of the sugars of the first, second and their alternate aspects of invention can be prepared as described below.

Other Sweeteners

In a third aspect, the present invention provides an amorphous sugar comprising (i) one or more sugar or alternative sweetener selected from the group consisting of lactose, maltose, trehalose, rice syrup, coconut sugar, monk fruit (dried or sourced from monk fruit juice or extract), agave, stevia, fermented stevia, maple syrup and combinations thereof, and (ii) a low GI drying agent. The amorphous sugar optionally further comprises one or more monosaccharide and/or disaccharide. Having developed stable amorphous powders of sucrose, the inventors of the present invention observed the health benefits associated with their products and progressed to developing similar amorphous products of other sugars/sweeteners, including those that are capable of spray drying such as lactose and monk fruit, with the intention of providing alternative sugars and sweetening ingredients to the food industry.

In an alternate third aspect, the present invention provides an amorphous sugar comprising (i) one or more sugar or alternative sweetener selected from the group consisting of sucrose, lactose, maltose, trehalose, rice syrup, coconut sugar, monk fruit (dried or sourced from monk fruit juice or extract), agave, stevia, fermented stevia, maple syrup and combinations thereof, and (ii) a low GI drying agent, with the proviso that when the sugar is sucrose, the drying agent is not whey protein isolate.

In an alternate third aspect, the present invention provides an amorphous sugar comprising (i) sugar or alternative sweetener selected from the group consisting of lactose, maltose, trehalose, rice syrup, coconut sugar, monk fruit, agave, stevia, fermented stevia, maple syrup, optionally sucrose, and combinations thereof, and one or more edible, high molecular weight, low GI drying agents, with the proviso that when the sugar is sucrose, the drying agent is not whey protein isolate.

In an alternate third aspect, the present invention provides an amorphous sugar comprising (i) sugar or alternative sweetener selected from the group consisting of lactose, maltose, trehalose, rice syrup, coconut sugar, monk fruit, agave, stevia, fermented stevia, maple syrup, optionally sucrose, and combinations thereof, and one or more edible, high molecular weight, low GI drying agents selected from the group consisting of lactose, protein, low GI carbohydrates, insoluble fibre, soluble fibre, lipids, natural intense sweeteners and/or combinations thereof, with the proviso that when the sugar is sucrose, the drying agent is not whey protein isolate.

In the third and alternate third aspects of the invention, it is preferred for the amorphous sugar to comprise relatively homogenous particles where each particle comprises both the drying agent and the one or more sugar/alternative sweetener.

In the third and alternative third aspects of the invention, the amorphous sugar optionally comprises an alternative sweetener. The alternative sweetener is optionally rice syrup, maple syrup, coconut sugar and/or monk fruit.

The sugar in the third and alternate third aspects of the invention is optionally selected from the group consisting of glucose, galactose, ribose, xylose, fructose, maltose, lactose, trehalose and combinations thereof.

The amorphous sweetener of the third and alternate third aspects of the invention optionally further comprises at least about 20 mg CE polyphenols/100 g carbohydrate and a low GI drying agent. The nature and amounts of polyphenols can be as described above for the first and second aspects of the invention. However, as the skilled person would be aware, where the one or more sweetener is already low GI, the polyphenols will not be needed for their GI lowering effect.

The amorphous sugar optionally has 40% to 95% w/w, 50% to 90% w/w or 50 to 80% w/w sugar/alternative sweetener.

The moisture content and flowability of the powder in the third and alternate third aspects of the invention can be as described for the first and second aspects of the invention.

In the third and alternative third aspects of the invention, the drying agent is as described below, with the proviso that when the sugar is lactose, the drying agent is not lactose.

Aerated versions of the sugars of the third and alternate third aspects of invention can be prepared as described below.

In the third and alternative third aspects of the invention, when the alternative sweetener is monk fruit, the drying agent is not also monk fruit.

The amorphous sugar of the third and alternate third aspects of the invention optionally remains a free flowing powder following 6, 12 or 18 months storage in ambient conditions.

Aerated Sugar

In some embodiments of the first, second, third and their alternative aspects of invention, the amorphous sugar is aerated. One advantage of an aerated sugar is that the surface area available to taste is increased while the ultimate quantity of sugar is decreased. This results in a sweeter taste but lower calories. The very small size of the air pocket or pores in the sugar means they cannot be felt in the mouth (by the tongue). This means the sugar retains a highly smooth mouth feel which is advantageous for many solid foods.

The aerated sugar of the invention is of particular use in the preparation of solid food, for example, by incorporation into a solid food matrix. Examples include chocolate, cakes and baked goods.

In a fourth aspect, the present invention provides an aerated amorphous sugar comprising one or more sugar or alternative sweetener selected from the group consisting of glucose, fructose, galactose, ribose, xylose, lactose, maltose, rice syrup, coconut sugar, monk fruit, agave, stevia, fermented stevia, maple syrup and combinations thereof, and a low GI drying agent. Optionally, the sugar is glucose and/or fructose.

In an alternate fourth aspect of the invention, the present invention provides an aerated amorphous sugar comprising one or more sugar or alternative sweetener selected from the group consisting of sucrose, glucose, fructose, galactose, ribose, xylose, lactose, maltose, rice syrup, coconut sugar, monk fruit, agave, stevia, fermented stevia, maple syrup and combinations thereof, and a low GI drying agent, wherein the sugar particles are between 1 and 100 μm in diameter (eg a D90 of 100 μm or less).

Optionally, in the fourth and alternate fourth aspects of the invention, the low GI drying agent is whey protein isolate, sunflower protein, xanthan gum, bagasse or combinations thereof. Preferably, the low GI drying agent is whey protein isolate optionally combined with a digestive resistant carbohydrate such as xanthan gum or bagasse.

In other embodiments, the low GI drying agent is a digestive resistant carbohydrate optionally with an aeration enhancer such as whey protein isolate, tocopherol phosphate and/or lecithin.

When whey protein isolate is combined with a digestive resistant carbohydrate, the ratio is optionally 20:1 to 5:1 w/w respectively.

In the fourth and alternate fourth aspects of the invention, it is preferred for the amorphous sugar to comprise relatively homogenous particles where each particle comprises both the drying agent and the one or more sugar/alternative sweetener.

In the fourth and alternate fourth aspects of the invention, it is preferred for the amorphous sugar to comprise relatively homogenous particles where each particle comprises both the drying agent and the one or more sugar/alternative sweetener.

The bulk density of the aerated amorphous sugars of the invention is about 0.25 to 0.7 g/cm³, about 0.3 to 0.7 g/cm³, 0.4 to 0.6 g/cm³or 0.45 to 0.55 g/cm³. The density is reduced 10 to 70%, 20 to 60% or 30 to 60% compared to traditional crystalline white sugar (sucrose).

In some embodiments of the aerated amorphous sugar of the invention, the sugar has up to 5% non-aerated particles, up to 10% non-aerated particles or up to 20% non-aerated particles. A non-aerated sugar of the invention may include some aerated particles. In some embodiments, the non-aerated amorphous sugar has up to 5% aerated particles, up to 10% aerated particles or up to 20% aerated particles.

An aerated sugar of a higher proportion of aerated particles may be prepared by sieving to remove the smaller non-aerated particles and retain the aerated particles. Using this method an aerated amorphous sugar with greater than 95% aerated particles, 99% aerated particles or about 100% aerated particles may be prepared.

Similarly, a non-aerated sugar with a higher proportion of non-aerated particle may be prepared by sieving to remove the larger aerated particles and retain the non-aerated particles. Using this method a non-aerated amorphous sugar with greater than 95% non-aerated particles, 99% non-aerated particles or about 100% non-aerated particles may be prepared. More aerated particles might be formed by agitation.

In some embodiments, the aerated sugar of the invention has non-agglomerated particles. In some embodiments the aerated sugar of the invention is openly aerated (in the sense that a reasonable proportion of the sugar particles (eg at least 20, 40, 60, or 80%) have an opened external surface rather than air pockets within a fully enclosed particle).

In some embodiments, the aerated sugar of the invention is both non-agglomerated and openly aerated.

The amorphous sugar of the fourth and alternate fourth aspects of the invention optionally remains a free flowing powder following 6, 12 or 18 months storage in ambient conditions.

The degree of aeration of aerated sugars of the invention can be increased by increasing the amount of whey protein isolate present. It is also possible to increase the degree of aeration by addition of lecithin and/or tocopherol phosphate. It is also possible to increase the amount of aeration by blowing air through the liquid feedstock before rapid drying.

Drying Agents

The drying agent is optionally a low GI carbohydrate such as corn starch and/or a protein. Alternatively, the edible drying agent is a protein, low GI carbohydrate, lipid and/or natural intense sweetener. Where the drying agent is of limited solubility a solubiliser can be used.

Suitable proteins include whey protein isolate, preferably bovine whey protein isolate, β-lactoglobulin, α-lactalbumin, serum albumin, pea protein, sunflower protein and hemp protein. Suitable proteins include whey protein isolate, preferably bovine whey protein isolate, β-lactoglobulin, α-lactalbumin, serum albumin, maltodextrin, pea protein, sunflower protein and hemp protein.

Optionally, the low GI drying agent is lactose.

Preferably, the low GI drying agent is digestion resistant. Suitable digestion resistant drying agents include hi-maize, fructo-oligosaccharide or inulin, bagasse, xanthan gum, or digestive resistant maltodextrin (ie a derivative of maltodextrin that resists digestion in the small intestine of healthy individuals, for example, because at least some of the glucose substituents have been converted to non-digestible forms) or its derivatives. The digestive resistant low GI drying agent is optionally a glucose polymer of 3 to 17 or 10 to 14 glucose units. The digestive resistant low GI drying agent may be a soluble or insoluble fibre or a combination thereof. One option for the digestive resistant low GI drying agent with insoluble fibre is bagasse. Xanthan gum is a soluble fibre suitable for use as a low GI drying agent.

Preferred drying agents include a digestive resistant carbohydrate or a digestive resistant starch such as hi-maize or the protein whey protein isolate or a combination thereof. One advantage of use of a digestive resistant starch is an improvement in anti-caking when using industrial quantities of the sugar.

Suitable lipids include phospholipids such as lecithin and phosphorylated vitamin E.

The natural intense sweeteners are intensely sweetening plant extracts or juices. These can be either liquid or dried. Suitable extracts and juices in liquid and dried forms are commercially available for stevia, monk fruit and blackberry leaf. In view of the monk fruit products prepared by the inventors, stevia and blackberry leaf versions of the sugars/sweeteners of the invention are expected to be successful.

Optionally, the drying agent for all aspects of the invention is monk fruit.

In some embodiments, the drying agent is a protein and a low GI carbohydrate combination, for example, whey protein isolate and hi-maize. A 1:1 w/w ratio of whey protein isolate and hi-maize is suitable.

In alternate embodiments, the drying agent is a protein and lipid combination, for example, whey protein isolate and lecithin. A 1:1 to 1:2 ratio of whey protein isolate to lecithin forms a stable powder. The drying agent is suitable for preparation as a free flowing amorphous powder. Therefore, while at least 5% w/w of the solids need to be drying agent to prepare a suitable amorphous solid, there is no maximum to the amount of drying agent (because the drying agent can be spray dried effectively alone).

Preferably, the molecular weight of the drying agent is higher than that of the reducing sugars glucose and fructose (ie about 180 g/mol). Optionally, the molecular weight of the drying agent is 200 g/mol to 70 kDa, 300 g/mol to 70 kDa, 500 g/mol to 70 kDa, 800 g/mol to 70 kDa, or 1 kDa to 70 kDa. Optionally, the drying agent is 10 kDa to 60 kDa, 10 kDa to 50 kDa, 10 kDa to 40 kDa, or 10 kDa to 30 kDa.

Optionally, the drying agent has 0 to 0.2% hygroscopicity at 50% relative humidity.

In some embodiments, the drying agent is a protein of 10 to 70 kDa (such as bovine whey protein isolate, β-lactoglobulin, α-lactalbumin, serum albumin or combinations thereof) and the ratio of sugar source and drying agent is 95:5 to 60:40 by solid weight. A product can be prepared with more drying agent but the taste profile of the above ratio was preferred. The skilled person would understand that higher amounts of high molecular weight drying agents with a relatively lower molecular weight will be needed to lower the glass transition temperature (Tg) of the amorphous sugar in the first, second and alternative aspects of the invention, where the Tg of the sugar is an issue. The skilled person would also understand that lower amounts of high molecular weight drying agents with a relatively higher molecular weight will be needed to lower the Tg of the amorphous sugar.

Optionally, the drying agent is from 5% to 60% w/w, 10 to 50% w/w or 20 to 50% w/w of the amorphous sugar/sweetener. Optionally, the drying agent is 5% to 60%, 5 to 40%, 5 to 35%, or 10 to 40% by weight. In some embodiments the drying agent is 5% to less than 40% w/w of the amorphous sugar.

In another embodiment, the present invention provides an amorphous sugar comprising 40% to 95% w/w sucrose, 0% to 4% w/w reducing sugars, at least about 20 mg CE polyphenols/100 g carbohydrate to about 1 g polyphenols CE/100 g carbohydrate and 5% to 60% w/w low GI drying agent, wherein the molecular weight of the drying agent is about 200 g/mol to about 70 kDa.

In another embodiment, the present invention provides an amorphous sugar comprising 40% to 95% w/w sucrose, 0% to 4% w/w reducing sugars, at least about 20 mg CE polyphenols/100 g carbohydrate to about 1 g polyphenols CE/100 g carbohydrate and 5% to 60% w/w low GI drying agent, wherein the molecular weight of the drying agent is about 200 g/mol to about 70 kDa and the drying agent is selected from the group consisting of digestive resistant carbohydrate or whey protein isolate or a combination thereof.

In another embodiment, the present invention provides an amorphous sugar comprising 40% to 95% w/w sucrose, 0% to 4% w/w reducing sugars, at least about 20 mg CE polyphenols/100 g carbohydrate to about 1 g polyphenols CE/100 g carbohydrate and 5% to 60% w/w low GI drying agent, wherein the molecular weight of the drying agent is about 200 g/mol to about 70 kDa and, wherein 10 g of the amorphous sugar of the invention has a glycaemic load of 10 or less or the amorphous sugar has a glucose base glycaemic index of less than 55.

Prebiotic Sugars

In a fifth aspect, the present invention provides a prebiotic amorphous sugar in accordance with any one of the amorphous sugars in the first to fourth aspects of the invention or their embodiments, wherein the low GI drying agent is a digestive resistant carbohydrate and the prebiotic amorphous sugar has a prebiotic effect when consumed. The low GI drying agent is optionally soluble fibre and/or insoluble fibre.

Suitable prebiotic drying agents include hi-maize, fructo-oligosaccharide or inulin, bagasse, xanthan gum, digestive resistant maltodextrin or its derivatives, a digestive resistant glucose polymer of 3 to 17 or 10 to 14 glucose units. The other features of the drying agent such as molecular weight, hygroscopicity and weight percentage drying agent versus sugar/sweetener are optionally as described above. Methods for testing the prebiotic effect of the prebiotic amorphous sugar are explained in Singaporean patent application SG 10201809224Y, titled “Compositions that reduce sugar bioavailability and/or have prebiotic effect”, a copy of which is incorporated into the body of this specification by reference.

Protein Sugars

In a sixth aspect, the present invention provides a protein containing amorphous sugar wherein the amorphous sugar is in accordance with any one of the first to fourth aspects of the invention or their embodiments and the low GI drying agent is a protein. The protein is optionally protein isolate, preferably bovine whey protein isolate, β-lactoglobulin, α-lactalbumin, serum albumin, pea protein, sunflower protein and/or hemp protein.

The other features of the drying agent such as molecular weight, hygroscopicity and weight percentage drying agent versus sugar/sweetener are optionally as described above.

Intense Sweeteners

In a seventh aspect, the present invention provides an amorphous sugar composition comprising an amorphous sugar in accordance with any one of the first to fourth aspects of the invention or their embodiments and a low GI drying agent, wherein the low GI drying agent is one or more natural intense sweeteners selected from the group consisting of stevia, monk fruit, blackberry leaf and their extracts, with the proviso that when the low GI drying agent is monk fruit or a monk fruit extract, the sugar/sweetener is not a monk fruit alternative sweetener. The protein is optionally 10 to 70 kDa.

The other features of the drying agent such as molecular weight, hygroscopicity and weight percentage drying agent versus sugar/sweetener are optionally as described above.

In one embodiment of the seventh aspect of the invention, the amorphous sugar contains polyphenols and optionally the sugar is sucrose and sourced from cane juice, beet juice or molasses. In these embodiments, the polyphenols and/or the caramel type flavour of the sugar source masks the metallic taste of the high intensity sweetener to either improve the taste of the sugar and/or allow an increased amount of high intensity sweetener while retaining palatability. An increased use of high intensity sweetener will allow for a reduced use of sugar in foods and beverages prepared using this embodiment of the invention.

Lipid Sugars

In an eight aspect, the present invention provides an amorphous sugar composition comprising an amorphous sugar in accordance with any one of the first to fourth aspects of the invention or their embodiments and a low GI drying agent, wherein the low GI drying agent is a phospholipid such as lecithin or phosphorylated vitamin E.

Options for All Sugars/Sweeteners of the Invention

The following section applies to all aspects, alternative aspects and embodiments of the amorphous sugar of the invention unless indicated otherwise.

The amorphous sugar is intended for use as a food and/or ingredient used in the preparation of food. The sugars, alternative sweeteners and drying agents used are always suitable for consumption (ie edible).

In all aspects of the invention comprising sucrose, unless otherwise specified the sucrose is optionally sourced from sugar cane and/or beet sugar. In all aspects of the invention comprising fructose, unless otherwise specified the fructose is optionally high fructose corn syrup.

The amorphous sugars of all aspects of the invention are optionally 40% to 95% w/w, 50% to 90% w/w or 50 to 80% w/w sugar or alternate sweetener.

Optionally, the amorphous sugars of all aspects of the invention have low hygroscopicity eg 0 to 0.2% at 50% relative humidity.

Optionally, anti-caking agents are added including but not limited to starch, calcium phosphate and/or magnesium stearate.

Optionally, the reducing sugars are 0% to 4% w/w, 0.1% to 3.5% w/w, 0% to 3% w/w, 0% to 2.5% w/w, 0.1% to 2% w/w of the amorphous sugar.

Optionally, the amorphous sugars of all aspects of the invention have a water activity (a_{w}) of less than 0.6, less than 0.4 or about 0.3.

In some embodiments, the amorphous sugar is low glycaemic or very low glycaemic.

Optionally, 10 g of the amorphous sugar of the invention has a glycaemic load (GL) of 10 or less, or 8 or less, or 5 or less. Calculation of glycaemic load of an amount of a food is explained in the detailed description below.

Optionally, the amorphous sugar of the invention has a glucose based GI of 54 or less or 50 or less. Optionally, the amorphous sugar has a glucose based GI of 54 or less and 10 g of the amorphous sugar has a glucose based GL of 10 or less.

Optionally, the amorphous sugar further comprises a flow agent and/or desiccant. A flow agent and/or desiccant is of particular assistance where the reducing sugars are above 2% w/w or above 3% w/w of the amorphous sugar.

The amorphous sugar is optionally a homogenous mixture of ingredients. Where larger drying agents are used, the amorphous sugar is optionally drying agent at its core with the drying agent coated by the sucrose and/or other smaller components of the amorphous sugar.

The amorphous sugar is comprised of particles. The particles are generally between 1 and 100 μm in diameter. The particles are optionally between 5 and 80 μm, 5 and 60 μm and 5 and 40 μm. A blend of smaller and larger particles is common, for example, a blend of particles less than 10 μm in diameter with particles of over 10 μm but less than 50 μm in diameter. It is also common for the aerated sugar of the invention (see below) to include some non-aerated particles immediately following its preparation.

While it is possible to coat the amorphous sugar particles, the particles are usually not coated.

Taste

In embodiments where the sugar is sucrose and it is sourced from cane juice, beet juice and/or molasses, the amorphous sugar of the invention has a desirable sensory profile, in particular, a taste that is sweeter than refined white sugar and/or a stronger caramel flavour than refined white sugar. Without being bound by theory, this is thought to occur either because the cane juice, beet juice and molasses sourced sugars are sweeter than essentially pure sugar and/or because the amorphous nature of the sugar allows for rapid tasting of the sugar compounds present in the amorphous sugar and/or because the aerated size of the sugar positions the sugar for increased contact with taste buds resulting in a stronger recognition of the sweetness.

Where the amorphous sugar of the invention includes whey protein isolate, the sugar optionally has a milkier taste than that for refined white sugar.

Reduced the Digestively Available Sugar or Calories/Increasing the Nutrition

The amorphous sugar of the invention is suitable for use as an ingredient in other foods or as a dietary supplement. The amorphous sugar of the invention can be used to reduce the sugar in a food system by 10% or more, 20% or more, 30% or more, or 40% or more, 55% or more or up to about 65%; relative to the use of traditional crystalline sugar in the food system. Optionally, the sugar in the food or beverage is reduced by 10-50% or 20-40%. The food system can be the sugar itself. This occurs because there is less free sugar in the amorphous sugar of the invention than in refined white sugar. Also, due to the sweetness of the amorphous sugar in embodiments of the invention where the sugar is sucrose and the sucrose is sourced from cane juice, beet juice and/or molasses, a less than a 1:1 sugar substitution may be required. See Example 12 for further detail.

The non-aerated amorphous sugar of the invention contains up to 15% less kilojoules and/or calories than white refined sugar, that is, it contains about 85% to 95% of the kilojoules and/or calories of white refined sugar.

The total kilojoule/calorie reduction for the amorphous sugar of the invention is optionally 5 to 40% or 10 to 30%, when the less than 1:1 substitution potential due to the increased sweetness of the amorphous sugar is considered.

For embodiments of the various aspects of invention where the sugar is sucrose and is sourced from cane juice, molasses and/or beet juice and there are at least 20 mg CE polyphenols/100 g carbohydrate present, the amorphous sugar has an improved nutritional profile compared to traditional white crystalline sugar. In these embodiments, the amorphous sugar optionally has one or more of:
5-9% (7%) of the recommended daily amount of sodium;
20-30% (23%) of the recommended daily amount of carbohydrates;
3-10% (4%) of the recommended daily amount of fibre;
10-50% (48%) of the recommended daily amount of protein;
50-100% (90%) of the recommended daily amount of calcium;
100-180% (160%) of the recommended daily amount of iron;
30-40% (35%) of the recommended daily amount of potassium;
50-80% (70%) of the recommended daily amount of magnesium;
25-35% (35%) of the recommended daily amount of zinc;
50-65% (60%) of the recommended daily amount of copper; and/or
200-400% (350%) of the recommended daily amount of manganese.

Where the low GI drying agent is whey protein isolate and the sugar is optionally sourced from cane juice, the amorphous sugar of the invention optionally has all of the above.

Method of Preparing Amorphous Sugars of the Invention

In another aspect, the present invention provides a method for preparing an amorphous sugar according to the first or alternate first aspects of the invention comprising (i) combining a liquid containing sucrose and polyphenols with at least one drying agent; and (ii) rapidly drying the mixture to produce the amorphous sugar.

Alternatively, the present invention provides a method for preparing an amorphous sugar according to the second or alternate second aspects of the invention comprising (i) combining a liquid containing one or more low molecular weight sugars and polyphenols with at least one drying agent; and (ii) rapidly drying the mixture to produce the amorphous sugar.

Alternatively, the present invention provides a method for preparing an amorphous sugar according to the third or alternate third aspects of the invention comprising (i) combining a liquid containing one or more sugars or alternative sweeteners and polyphenols with at least one drying agent; and (ii) rapidly drying the mixture to produce the amorphous sugar.

Surprisingly an aerated sugar according to the invention can also be prepared by (i) mixing a liquid containing sucrose and polyphenols with at least one drying agent; and (ii) rapidly drying the mixture to produce the amorphous sugar. What is surprising is that very mild mixing by hand is effective as it was expected that air would need to be introduced into the feedstock to achieve the aeration.

In one embodiment, an aerated sugar according to the invention can also be prepared by (i) mixing a liquid containing sucrose and polyphenols with at least one drying agent; and (ii) rapidly drying the mixture to produce the amorphous sugar, wherein no additional air is pumped into the feedstock prior to rapid drying.

In another embodiment, an aerated sugar according to the invention can also be prepared by (i) mixing a liquid containing sucrose and polyphenols with at least one drying agent; and (ii) rapidly drying the mixture to produce the amorphous sugar, wherein the mixing does not create a bubbled feedstock prior to rapid drying.

In an alternative embodiment, an aerated sugar according to the invention can be prepared by (i) mixing a liquid containing sucrose and polyphenols with at least one drying agent; and (ii) rapidly drying the mixture to produce the amorphous sugar, wherein the mixing creates a bubbled feedstock prior to rapid drying but no additional air is pumped into the feedstock prior to rapid drying.

Optionally the rapid drying uses a spray drier. Optionally, the spray drier is a counter current spray drier. Alternatively, the spray drier is a co-current spray drier.

The liquid is optionally selected from the group consisting of cane juice, beet juice and molasses. The liquid is preferably cane juice and/or molasses. Optionally, the liquid is prepared with (or diluted/concentrated until it has) 5 to 30%, 10 to 25%, 15 to 20% or 20% w/w total solids. Sugarcane juice is optionally at least 60 Brix (ie 60 g sucrose in 100 g solution). Results vary depending upon the sugarcane variety.

The liquid and drying agent are both optionally 0.1 micron filtered. The liquid and drying agent are combined. The liquid and drying agent has 20 mg CE polyphenols/100 g carbohydrate to 1 g CE polyphenols/100 g carbohydrate. The polyphenol content is optionally adjusted by adding additional polyphenols (or reducing polyphenols by dilution) prior to drying.

The inlet air temperature for the spray drier is optionally 140° C. to 200° C., 160° C. to 200° C., 140° C. to 180° C., 140° C. to 160° C. or 160° C. to 180° C.

The outlet air temperature for the spray drier is 70° C. to 90° C., 75° C. to 85° C. or 75° C. to 80° C.

Glucose oxidase may be added to the liquid before drying to decrease free glucose if required.

One advantage of preparing a sugar by spray drying is that the processing is inexpensive. Other low cost drying methods may also be useful including fluidized bed drying, low temperature vacuum drying and ring drying. It is also beneficial that some of the vitamins, minerals and phytochemical compounds naturally in the sugar are retained so the sugar retains nutritional value and is not a “hollow nutrient”.

One advantage of the spray dried amorphous sugar of the present invention (for embodiment using cane juice, beet juice or molasses as a sucrose source) is that the spray dried sugar is utilising a former sugar waste stream, molasses, to increase sugar production or utilising a less refined product cane juice to increases production and improve efficiency when compared to preparation of traditional crystalline sugars.

Foods/Beverages

The invention also relates to foods or beverages comprising one or more amorphous sugars according to any aspect or embodiment of the invention.

For example, the present invention provides a chocolate containing an aerated amorphous sugar of the invention. The chocolate coats the aerated amorphous sugar particles coated with chocolate to form particles of up to about 100 μm in diameter. A chocolate with particles of smaller size, eg less than 30 μm in diameter or less than 20 μm in diameter, may be prepared by sieving the aerated amorphous sugar to remove larger particles. Similarly, smaller particles could be removed if desired.

In another aspect, the present invention provides a baked good containing an aerated amorphous sugar of the invention. The baked good is optionally a biscuit, cake or muffin.

In another aspect, the present invention provides a beverage containing an amorphous sugar or alternative sweetener according to any aspects, alternate aspect or embodiment of the invention. Optionally, the alternative sweetener is monk fruit or low GI drying agent is an intense sweetener such as monk fruit.

In yet another aspect, the present invention provides a composition comprising (i) an amorphous sugar or amorphous alternative sweetener according to any aspects, alternate aspect or embodiment of the invention and milk powder, coffee and/or chocolate. These compositions are suitable for the preparation of beverages (ie for combining with milk or water to prepare coffee, chocolate or mocha drinks) or as an ingredient in foods, for example, baked goods. Optionally, the amorphous sugar or alternative sweetener is a prebiotic sugar or alternative sweetener according to the invention.

In the foods, chocolate, baked goods and composition described in this section, it is preferred that where an aerated sugar of the invention was used, that the aerated sugar has retained its aeration throughout the preparation of the food and is present in the food in its aerated form. This allows to additional bulking of the food, which in turn can allow for a sugar reduction in the food. Without being bound by theory, this is thought to be effective because a subject consuming the food only tastes the sugar on the surface of the sugar particle. The sugar from an amorphous sugar is tasted readily while the sugar from a crystalline sugar is tasted more slowed due to the time taken for the sugar compound to be released from the crystalline structure. The sugar in the centre of the particle is never tasted. Therefore, if part of the centre of the sugar particle is protein or fibre or air, the consumer of the particle may not register the difference but the sweetness of the sugar particle may be retained or even improved and the bulking effect of the sugar may also be retained or even improved.

Lowering the GI/GL of a Food/Beverage

In another aspect, the present invention provides a method of lowering the GR, GI and/or GL of a food or beverage comprising using a low GI and/or low GL amorphous sugar of this invention to prepare a food/beverage. It will be apparent to the skilled person that where the amorphous sugar of the invention contains an amount of sucrose (and other sugars) and an amount of a low GI drying agent, the GI of the amorphous sugar will vary depending on the proportion of sugar to low GI drying agent. The GL will further vary with the amount of sugar consumed.

In another aspect, the present invention provides a method of lowering the GI of a meal, in particular a carbohydrate containing meal, comprising consuming a dietary supplement up to 30 minutes before, during or up to 30 minutes after eating the meal, wherein the supplement comprises the amorphous sugar of the invention.

Method of Preparing Food

In another aspect, the present invention provides a method of preparing a chocolate or baked good in which the traditional sugar in the recipe has been substituted by a sugar according to the invention (for example an aerated sugar of the invention), wherein (i) the non-sugar ingredients of the chocolate or baked good are combined and (ii) the amorphous sugar is mixed with the non-sugar ingredients immediately prior to baking/setting.

Alternatively, the present invention provides a method of preparing a chocolate or baked good in which the traditional sugar in the recipe has been substituted by a sugar according to the invention (for example an aerated sugar of the invention), wherein (i) half of the total amorphous sugar required is added when the traditional sugar would have been added, and (ii) the remainder of the amorphous sugar is mixed with the other ingredients immediately prior to baking/setting.

The chocolate or baked good optionally comprises amorphous sugar particles of less than 30 μm or less than 20 μm in diameter.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a diagram of a typical counter current spray dryer (G=gas/air, F=feed, P=powder, S=spray)

FIG. 2 depicts moisture content of 80:20 cane juice to whey protein isolate vs average drying chamber temperature for samples 2 to 4 of Table 6.

FIG. 3A is a scanning electron microscope (SEM) image of the 80:20 CJ:WPI % solids amorphous sugar, wherein the scale bar corresponds to 100 μm.

FIG. 3B is a scanning electron microscope (SEM) image of the 70:30 CJ:WPI % solids amorphous sugar, wherein the scale bar corresponds to 100 μm.

FIG. 4 graphs the results of an in vitro Glycemic Index Speed Test (GIST) on the 90:10 CJ:WPI sugar from Example 8 showing the sugar is low glycaemic.

FIG. 5A charts the results of a study on the effect of polyphenol content or polyphenol plus reducing sugar content on the GI of sucrose in the form of traditional refined white sugar. 30, 60 and 120 mg CE polyphenol/100 g carbohydrate content was tested. The GI for sucrose with 60 mg CE polyphenol/100 g carbohydrate was shown to be about 15. Adding 0.6% w/w reducing sugars (1:1 glucose to fructose) to the sucrose with 30 mg CE polyphenols/100 g carbohydrate raised the GI from 53 to 70. Adding 0.6% w/w reducing sugars (1:1 glucose to fructose) to the sucrose with 60 mg CE polyphenols/100 g carbohydrate raised the GI from 15 to 29. Adding 1.2% w/w reducing sugars (1:1 glucose to fructose) to the sucrose with 120 mg CE polyphenols/100 g carbohydrate increased the GI from 65 to 75. The presence of reducing sugar consistently increased the GI.

FIG. 5B graphs the GI of several samples from Table 10 in Example 9.

FIG. 6 depicts the sensory profile of the 90:10, 80:20 and 70:30 CJ:WPI % solids amorphous sugars from Example 8. The 90:10 and 80:20 sugars are sweeter than refined white sugar, while the 70:30 is equivalently sweet. The 90:10 and 80:20 sugars have a caramel taste. The 80:20 and 70:30 sugars have a milky taste.

FIG. 6A-E are SEM images of the aerated sugars of Example 11, wherein the scale bar in FIG. 6A corresponds to 20 μm, the scale bar in FIG. 6B corresponds to 20 μm, the scale bar in FIG. 6C corresponds to 10 μm, the scale bar in FIG. 6D corresponds to 10 μm and the scale bar in FIG. 6E corresponds to 20 μm.

FIG. 6 shows that in general, the particle size is not evenly distributed. Some particles are about 60 μm, others are less than 10 μm. A great number of porous particles were detected, especially from the chipped particle powders.

FIG. 7 shows an image of 3 g of white crystal sugar and 3 g of the aerated amorphous sugar prepared according to this Example 11. The image illustrates the difference in bulk density. The bulk density of the white crystal sugar was calculated to be approximately 0.88 g/cm³. The bulk density the aerated amorphous sugar prepared according to this Example 11 was found to be approximately 0.47 g/cm³.

FIG. 8A-D are SEM images that show the chocolate of Example 13 prepared with sugar crystals, wherein the scale bar in FIG. 8A corresponds to 10 μm, the scale bar in FIG. 8B corresponds to 10 μm, the scale bar in FIG. 8C corresponds to XXX μm and the scale bar in FIG. 8D corresponds to 20 μm.

The sample indicates solid chocolate with tactile sugar crystals.

FIG. 8E-H are SEM images that show the chocolate of Example 13 prepared with the aerated amorphous sugar, wherein the scale bar in FIG. 8E corresponds to 10 μm, the scale bar in FIG. 8F corresponds to 10 μm, the scale bar in FIG. 8G corresponds to 10 μm and the scale bar in FIG. 8H corresponds to 10 μm.

These images show that the aerated sugar particles remain intact in the chocolate product and have not lost their aeration during food preparation. While the aeration is less evident due to a layer of fat coating the sugar, the particle remains aerated as it retains its pre-processing size and shape.

FIG. 9A-C are SEM images of product 1 from Table 12 (comprising rice syrup), wherein the scale bar in FIG. 9A corresponds to 500 μm, the scale bar in FIG. 9B corresponds to 50 μm and the scale bar in FIG. 9C corresponds to 30 μm.

FIG. 9A-C shows that in general, the particle size is reasonably evenly distributed, with most particles ranging from about 25 μm to about 50 μm in size. Porosity was observed.

FIG. 9D-E show SEM images of product 2 from Table 12 (comprising coconut sugar), wherein the scale bar in FIG. 9D corresponds to 300 μm and the scale bar in FIG. 9E corresponds to 20 μm.

FIG. 9D-E shows that in general, the particle size is reasonably evenly distributed, with most particles ranging from about 20 μm to about 55 μm in size. Porosity was observed.

FIG. 9F-G show SEM images of product 3 from Table 12 (comprising monk fruit), wherein the scale bar in FIG. 9F corresponds to 30 μm and the scale bar in FIG. 9G corresponds to 10 μm.

FIG. 9F-G shows that in general, the particle size is not evenly distributed. Some particles are about 100 μm, others are around 10 μm. Porosity was observed.

FIG. 9H-I show SEM images of product 4 from Table 12 (comprising maple syrup), wherein the scale bar in FIG. 9H corresponds to 300 μm and the scale bar in FIG. 9I corresponds to 20 μm.

FIG. 9H-I shows that in general, the particle size is reasonably evenly distributed, with most particles ranging from about 30 μm to about 60 μm in size. Porosity was observed.

FIG. 9J-K show SEM images of product 6 from Table 12 (comprising bagasse), wherein the scale bar in FIG. 9J corresponds to 100 μm and the scale bar in FIG. 9K corresponds to 10 μm.

FIG. 9J-K shows that in general, the particle size is reasonably evenly distributed, with most particles ranging from about 20 μm to about 30 μm in size. Porosity was observed.

FIG. 9L-M show SEM images of product 7 from Table 12 (comprising sunflower protein), wherein the scale bar in FIG. 9L corresponds to 200 μm and the scale bar in FIG. 9M corresponds to 50 μm.

FIG. 10 shows SEM images of the butter cookie prepared according to Example 15, wherein the scale bar in FIG. 10A corresponds to 10 μm and the scale bar in FIG. 10B corresponds to 10 μm.

These images show that the aerated sugar particles remain intact in the cookie product and have not lost their aeration during food preparation. While the aeration is less evident due to a layer of fat coating the sugar, the particle remains aerated as it retains its pre-processing size and shape.

FIG. 11 shows SEM images of the vanilla muffin prepared according to Example 15, wherein the scale bar in FIG. 11A corresponds to 20 μm and the scale bar in FIG. 11B corresponds to 10 μm.

These images show that the aerated sugar particles remain intact in the muffin product and have not lost their aeration during food preparation. While the aeration is less evident due to a layer of fat coating the sugar, the particle remains aerated and it retains its pre-processing size and shape.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

Further aspects of the present invention and further embodiments of the aspects described in the preceding paragraphs will become apparent from the following description, given by way of example.

All of the patents and publications referred to herein are incorporated by reference in their entirety.

For purposes of interpreting this specification, terms used in the singular will also include the plural and vice versa.

One skilled in the art will recognize many methods and materials similar or equivalent to those described herein, which could be used in the practice of the present invention. The present invention is in no way limited to the methods and materials described.

The inventors of the present invention have developed an amorphous sugar comprising sucrose, at least about 20 mg CE polyphenols/100 g carbohydrate and a low GI drying agent. The sugar is an alternative to traditional sugars that could increase sugar supply. It is also reduces the GR, GI and/or GL of foods, or amounts of foods, it is included in for better health.

The inventors of the present invention have developed a new prebiotic sugar. As many popular foods, particularly foods with high sugar content, have a less than ideal impact on to the gastro-intestinal microbiome, the preparation of prebiotic sugars is a highly significant advance. The prebiotic sugars of the invention provide sugar substitutes that avoid one of the less desirable aspects of sugar and introduce a desirable prebiotic effect into sugars that will increase the health benefits of foods comprising the prebiotic sugars.

The term “aerated” refers to including air. In particular, in the context of this invention an aerated particle is one that includes air pockets or air bubbles ie is porous in nature.

The term “amorphous” refers to a solid that is largely amorphous, that is, largely without crystalline structure. For example, the solid could be 80% or more amorphous, 90% or more amorphous, 95% or more amorphous or about 100% amorphous.

The term “bagasse” refers to sugar fibre either from sugar cane or sugar beet. It is the fibrous pulp left over after sugar juice is extracted. Bagasse products are commercially available, for example, Phytocel is a sugar cane bagasse product sold by KFSU.

The term “drying agent” refers to an agent that is suitable for rapid drying with sucrose to achieve a dry powder as opposed to the sticky powder achieved is sucrose is dried alone.

The term “high molecular weight drying agent” refers to a drying agent with a molecular weight above that of sucrose, for example, about the molecular weight of lactose or higher.

The term “low glycaemic” refers to a food with a glucose based GI of 55 or less.

The term “very low glycaemic” refers to a food with a glucose-based GI of less than half the upper limit of low GI (ie the GI is in the bottom half of the low GI range).

The term “sugar” refers to a solid that contains one or more low molecular weight sugars (monosaccharides) such as glucose or disaccharides such as sucrose etc. In the context of the invention, the sugars referred to are edible sugars used in the production of food. The amorphous sugars of the invention could be spray dried cane juice or molasses but could also be spray dried fruit juice.

The term “reducing sugar” refers to any sugar that is capable of acting as a reducing agent. Generally, reducing sugars have a free aldehyde or free ketone group. Glucose, galactose, fructose, lactose and maltose are reducing sugars. Sucrose and is not a reducing sugar.

The term “phytochemical” refers generally to biologically active compounds that occur naturally in plants.

The term “polyphenol” refers to chemical compounds that have more than one phenol group. There are many naturally occurring polyphenols and many are phytochemicals. Flavonoids are a class of polyphenols. Polyphenols including flavonoids naturally occur in sugar cane. In the context of the present invention the polyphenols that naturally occur in sugar cane are most relevant. Polyphenols in food are micronutrients that are of interest because of the role they are currently thought to have in prevention of degenerative diseases such as cancer, cardiovascular disease or diabetes.

The term “refined white sugar” refers to fully processed food grade white sugar that is essentially sucrose with minimal reducing sugar content and minimal phytochemicals such as polyphenols or flavonoids.

The term “massecuite” refers to a dense suspension of sugar crystals in the mother liquor of sugar syrup. This is the suspension that remains after concentration of the sugar juice into a syrup by evaporation, crystallisation of the sugar and removal of molasses. The massecuite is the product that is washed in a centrifuge to prepare bulk sugar crystals.

The term “sugar juice” refers to the syrup or liquid extracted from sugar-rich plant feedstocks, such as the juice extracted following crushing/pressing sugar cane or the liquid exiting a diffuser during the processing of sugar beets.

The term “cane juice” or “sugar cane juice” refers to the syrup extracted from pressed and/or crushed peeled sugar cane. Ideally sugar cane juice is at least 60 Brix.

The term “beet juice” refers to the liquid exiting a diffuser after the beet roots have been sliced into thin strips called cossetes and passed into a diffuser to extract the sugar content into a water solution.

The terms “efficacious” or “effective amount” refer to an amount that is biologically effective. In this context, one example is an effective amount of polyphenols in the sugar particles to achieve a low GI sugar, ie, a sugar that causes a low increase in blood sugar levels once consumed such that an insulin response is avoided.

The term “hi-maize” or “high amylose maize starch” refers to a resistant starch, ie a high molecular weight carbohydrate starch that resists digestion and behaves more like a fibre. Hi-maize is generally made from high amylose corn. There are 2 main structural components of starch; amylose—a linear polymer of glucose residues bound via α-D-(1,4)-glycosidic linkages and amylopectin—a highly branched molecule comprising α-D-(1,4)-linked glucopyranose units with α-D-(1,6)-glycosidic branch points. Branch points typically occur between chain lengths of 20 to 25 glucose units, and account for approximately 5% of the glycosidic linkages. Normal maize starch typically consists of approximately 25 to 30% amylose and 75 to 80% amylopectin. High amylose maize starch contains 55 to >90% amylose. The structure for amylose is (with an average degree of polymerisation of 500):

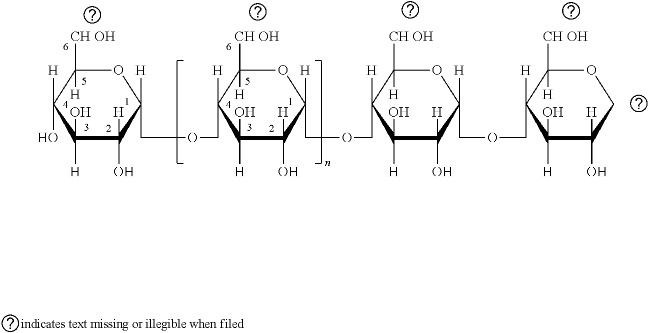

The structure for amylopectin is (with an average degree of polymerisation of 2 million):

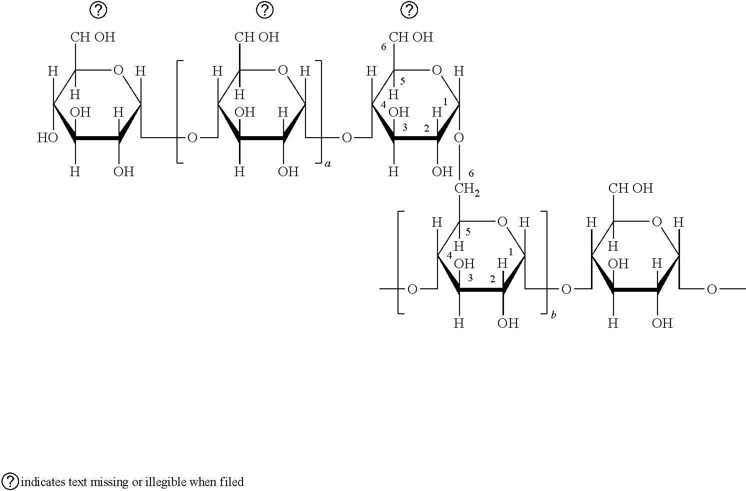

The term “inulin” refers to one or more digestive resistant high molecular weight polysaccharides having terminal glucosyl moieties and a repetitive frucosyl moitey linked by β(2,1) bonds. Generally, inulin has 2 to 60 degrees of polymerisation. The molecular weight varies but can be for example about 400 g/mol, about 522 g/mol, about 3,800 g/mol, about 4,800 g/mol or about 5,500 g/mol. Where there the degree of polymerisation is 10 or less the polysaccharide is sometimes referred to as a fructooligosaccharide. The term inulin has been used for all degrees of polymerisation in this specification. Inulin has the following structure:

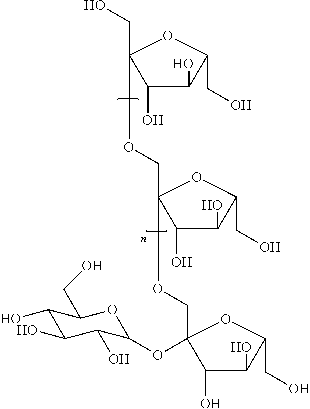

One option is to use Orafti Inulin with a molecular weight of 522.453 g/mol.

The term “dextrin” refers to a dietary fibre that is a D-glucose polymer with α-1,4 or α-1,6 glycosidic bonds. Dextrin can be cyclic ie a cyclodextrin. Examples include amylodextrin and maltodextrin. Maltodextrin is typically a mixture of chains that vary from 3 to 17 glucose units long. The molecular weight can be for example 9,000 to 155,000 g/mol.

The term “digestive resistant dextrin derivatives” refers to a dextrin modified to resist digestion. Examples include polydextrose, resistant glucan and resistant maltodextrin. Fibersol-2 is a commercial product from Archer Daniels Midland Company that is digestion resistant maltodextrin. An example structure is:

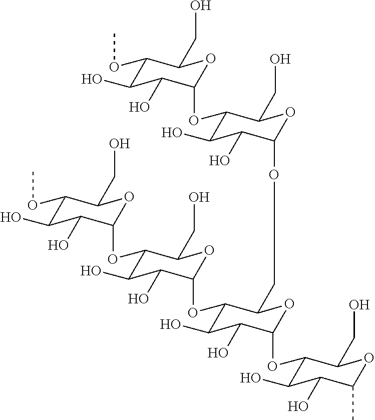

The term “whey protein isolate” refers to proteins isolated from milk, for example, whey can be produced as a by-product during the production of cheese. The whey proteins may be isolated from the whey by ion exchangers or by membrane filtration. Bovine whey protein isolate is a common form of whey protein isolate. Whey protein isolate has four major components: β-lactoglobulin, α-lactalbumin, serum albumin, and immunoglobulins. β-lactoglobulin has a molecular weight of 18.4 kDa. α-lactalbumin has a molecular weight of 14,178 kDa. Serum albumin has a molecular weight of 65 kDa. The immunoglobulin (Ig) in placental mammals are IgA, IgD, IgE, IgG and IgM. A typical immunoglobulin has a molecular weight of 150 kDa.

The term “high intensity sweetener” refers to either a natural or an artificial sweetener that has a higher sweetness than sucrose by weight ie less of the high intensity sweetener than the amount of sucrose is needed to achieve a similar sweetness level. Sucrose has a sweetness of 1 on the sucrose relative sweetness scale. For example, monk fruit extract has a sweetness value of about 150 to 300 sweeter than sucrose, blackberry leaf extract is about 300 times sweeter than sucrose and stevia is about 200-300 times sweeter than sucrose. Monk fruit extract, blackberry leaf extract and stevia are examples of natural high intensity sweeteners because they are sourced from plant by extraction and/or purification.

The term “stevia” refers to a sweetener prepared from the stevia plant including steviol glycosides such as Steviol, Steviolbioside, Stevioside, Rebaudioside A (RA), Rebaudioside B (RB), Rebaudioside C (RC), Rebaudioside D (RD), Rebaudioside E (RE), Rebaudioside F (RF), Rubusoside and Dulcoside A (DA) or a sweetener comprising the highly purified rebaudioside A extract approved by the FDA and commonly marketed as “stevia”.

The term “prebiotic” refers to a food ingredient that stimulates the growth and/or activity of one or more beneficial gastrointestinal bacteria. Prebiotics may be non-digestible foods or of low digestibility. A prebiotic can be a fibre but not all fibres are prebiotic. Oligosaccharides with a low degree of polymerisation ie ≤5 are thought to better stimulate bacteria concentration than oligosaccharides with higher degree of polymerisation.

The term “water activity” (a_{w}) is a measure of the partial vapor pressure of water in a substance divided by the standard state partial vapour pressure of water. Water migrates from areas of high a_{w} to areas of low a_{w}. Water activity is measured to determine shelf-stable foods. A water activity of 0.6 or less is preferred for foods and food ingredients of this type to inhibit mould and bacterial growth.

Particle size distribution can be defined using D values. A D90 value describes the diameter where ninety percent of the particle distribution has a smaller particle size and ten percent has a larger particle size.

Glycaemic Response (GR)

GR refers to the changes in blood glucose after consuming a carbohydrate-containing food. Both the GI of a food and the GL of an amount of a food are indicative of the glycaemic response expected when food is consumed.

GI

The glycaemic index is a system for classifying carbohydrate-containing foods according to the relative change in blood glucose level in a person over two hours after consuming that a food with a certain amount of available carbohydrate (usually 50 g). The two hour blood glucose response curve (AUC) is divided by the AUC of a glucose standard, where both the standard and the test food must contain an equal amount of available carbohydrate. An average GI is usually calculated from data collected from 10 subjects. Prior to a test the person would typically have undergone a twelve hour fast. The glycaemic index provides a measure of how fast a food raises blood-glucose levels inside the body. Each carbohydrate containing food has a GI. The amount of food consumed is not relevant to the GI. A higher GI generally means a food increases blood-glucose levels faster. The GI scale is from 1 to 100. The most commonly used version of the scale is based on glucose. 100 on the glucose GI scale is the increase in blood-glucose levels caused by consuming 50 grams of glucose. High GI products have a GI of 70 or more. Medium GI products have a GI of 55 to 69. Low GI products have a GI of 54 or less. These are foods that cause slow rises in blood-sugar.

Those skilled in the art understand how to conduct GI testing, for example, using internationally recognised GI methodology (see the Joint FAO/WHO Report), which has been validated by results obtained from small experimental studies and large multi-centre research trials (see Wolever et al 2003).

In vitro GI testing is now also available, see Example 4.

GL

Glycaemic load is an estimate of how much an amount of a food will raise a person's blood glucose level after consumption. Whereas glycaemic index is defined for each type of food, glycaemic load is calculated for an amount of a food. Glycaemic load estimates the impact of carbohydrate consumption by accounting for the glycaemic index (estimate of speed of effect on blood glucose) and the amount of carbohydrate that is consumed. High GI foods can be low GL. For instance, watermelon has a high GI, but a typical serving of watermelon does not contain much carbohydrate, so the glycaemic load of eating it is low.

One unit of glycaemic load approximates the effect of consuming one gram of glucose. The GL is calculated by multiplying the grams of available carbohydrate in the food by the food's GI and then dividing by 100. For one serving of a food, a GL greater than 20 is high, a GL of 11-19 is medium, and a GL of 10 or less is low.

Cane Juice

Cane juice contains all the naturally occurring macronutrients, micronutrients and phytochemicals present in the syrup extracted from pressed and/or crushed peeled sugar cane that are normally removed in white refined sugar, which is 99.9% sucrose.

Molasses

Is a viscous by-product of sugar preparation, which is separated from the crystallised sugar. The molasses may be separated from the sugar at several stages of sugar processing. Molasses contains the same compounds as cane juice but is a more highly concentrated source of phytochemicals.

Spray Drying and Other Drying Methods

Spray drying operates on the principle of convection to remove the moisture from the liquid feed, by intimately contacting the product to be dried with a stream of hot air. The spray drying process can be broken down into three key stages: atomisation of feedstock, mixing of spray and air (including evaporation process) and the separation of dried product from the air. Other appropriate drying methods include fluidized bed drying, ring drying, freeze drying and low temperature vacuum dehydration.

Atomisation

In order to ensure that the particles to be dried have the maximum surface area available to contact the hot air stream, the liquid feed is often atomised, producing very fine droplets ultimately leading to more effective drying. There are several atomiser configurations that exist, the most common being the wheel-type, pneumatic and nozzle atomisers.

A pneumatic high pressure nozzle atomiser was used for the experiments described below.

Evaporation and Separation

The second stage of the spray drying process involves the evaporation of moisture by using hot gases which flow around the surface of the particles/droplets to be dried.

There are notably three different types of air-droplet contacting configurations that exist: co-current, counter-current and mixed flow, all of which have differing applications depending on the product to be dried.

Both co-current and counter-current drying chambers are able to be used for heat sensitive materials, however the use of mixed-flow drying chambers is restricted to drying materials that are not susceptible to quality degradation due to high temperatures.

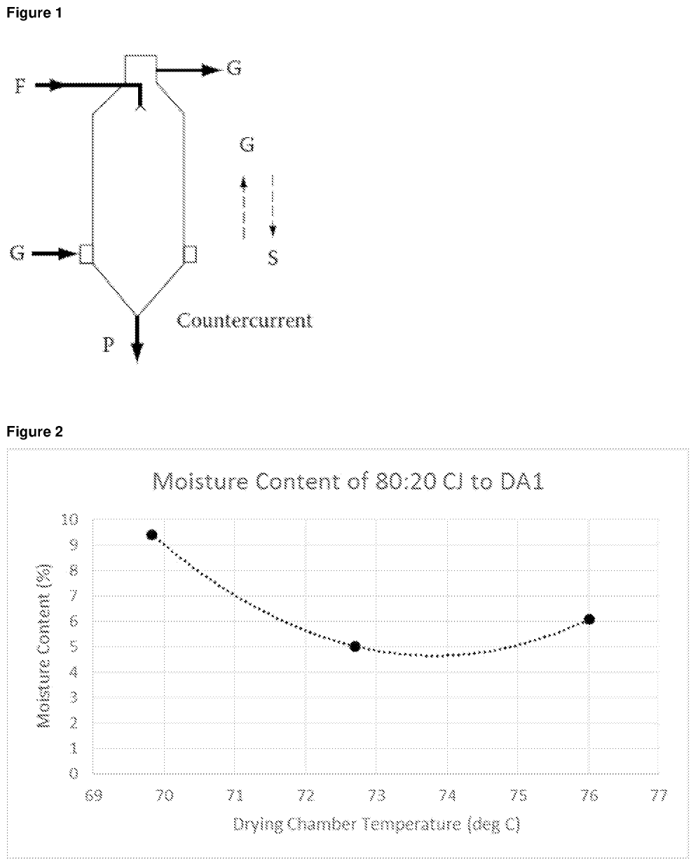

Representations of typical counter-current and co-current dryer setup is shown below in FIG. 1.

The final stage of the spray drying process is the separation of the powder from the air stream. The dry powder collects at the base of the drying chamber before it is discharged or manually collected.

Glass Transition Temperature

The glass transition temperature (Tg) is the substance-specific temperature range at which a reversible change occurs in amorphous materials from the solid, glassy state to the supercooled liquid state or the reverse. The glass transition temperature becomes very important for the production of dried products, particularly in relation to the processing and storage stages of manufacture. The glass transition temperature of the powders can be determined via differential scanning calorimetry (DSC).

ICUMSA

ICUMSA is a sugar colour grading system. Lower ICUMSA values represent less colour. ICUMSA is measured at 420 nm by a spectrophotometric instrument such as a Metrohm NIRS XDS spectrometer with a ProFoss analysis system. Currently, sugars considered suitable for human consumption, including refined granulated sugar, crystal sugar, and consumable raw sugar (ie brown sugar), have ICUMSA scores of 45-5,000.

Prebiotic Testing

The prebiotic effect of the sugars and alternate sweeteners of the invention can be tested using the Triskelion TNO Intestinal Model 2. This in an in vitro model of the gastrointestinal tract including a model colon with a variety of bacterial species presence such that an increase in probiotic following consumption of the prebiotic can be measured.

High Intensity Sweeteners

A natural low calorie sweetener, stevia, has also been developed and approved for use in many countries. Stevia is a high intensity sweetener meaning that one gram is much sweeter than one gram of sugar. Stevia has been used, in combination with sucrose, in several commercial products. However, consumers consider stevia to have an undesirable metallic aftertaste.

Monk fruit extract and blackberry leaf extract are alternative natural high intensity sweeteners.

Monk Fruit Extract and Blackberry Leaf Extract

Monk fruit extract is of interest because it has zero glycaemic index, contains no calories and is a natural product. The sweetness is from the mogrosides which make up about 1% of monk fruit. Monk fruit extract is being cultivated in New Zealand by BioVittoria. Monk fruit extract is also heat stable and has a long shelf life making it suitable for cooking and storage.

Monk fruit extract is prepared by crushing monk fruit and extracting the juice in water. The extract is filtered and the triterpene glycosides called mogrosides collected. It is sold in both liquid and powdered form. The extract is often combined with a bulking agent in powdered form.

Monk fruit extract costs more than stevia but has a less intense metallic after taste than stevia.

The sweetness index for monk fruit extract is up to 300 ie it is up to 300 times sweeter than sucrose depending on the specific extract used.

Blackberry leaf extract is similarly prepared by extracting blackberry leaves.

Stevia can be prepared by extracting stevia leaves but it is often further purified to improve the proportion of Rebaudioside A to other components with less beneficial flavour profiles.

Both monk fruit extract and blackberry extract are available from Hunan NutraMax Inc, F25, Jiahege Building, 217 Wanjiali Road, Changsha, China 410016, http://www.nutra-max.com/

### REFERENCES

International patent application no PCT/AU2017/050782.

Jaffé, W. R., (2012) Sugar Tech, 14:87-94.

Joint FAO/WHO Report. Carbohydrates in Human Nutrition. FAO Food and Nutrition. Paper 66. Rome: FAO, 1998.

Kim, Dae-Ok, et al (2003) Antioxidant capacity of phenolic phytochemicals from various cultivars of plums. Food Chemistry, 81, 321-26.

Singaporean patent application no SG 10201807121Q.

Wolever T M S et al. (2003) Determination of the glycemic index values of foods: an interlaboratory study. European Journal of Clinical Nutrition, 57:475-482.

A copy of each of these is incorporated into this specification by reference.

### EXAMPLES

### Example 1

### Spray-Dried Cane Juice and Molasses with Various Low GI HMWCs

Solutions were prepared according to Table 1. Spray drying solutions were created at a ratio of 1 g of HMWC to 1 g of sucrose, in the form of either molasses or cane juice. These solutions were then made up to a concentration of 20% total solid and sprayed in 400 or 500 ml quantities.

TABLE 1
solutions for spray drying

| | | | % w/w | | |
|---|---|---|---|---|---|
| Number | Sample | Ratio | Total Solids | Viscosity | Solubility |
| 1, 2 & 3 | Inulin + Cane Juice | 1:1 | 20 | <21 Mpas | Yes* |
| 4 | Inulin + Molasses | 1:1 | 20 | <21 Mpas | Yes* |
| 5 | Hi Maize + Cane Juice | 1:1 | 20 | <21 Mpas | No |
| 6 | Hi Maize + Molasses | 1:1 | 20 | <21 Mpas | No |
| 7 | Calcium Phosphate + Cane Juice | 1:1 | 20 | <21 Mpas | No |
| 8 | Calcium Phosphate + Molasses | 1:1 | 20 | <21 Mpas | No |
| 9 | Dextrin + Cane Juice | 1:1 | 20 | <21 Mpas | Yes |
| 10 | Dextrin + Molasses | 1:1 | 20 | <21 Mpas | Yes |
| 11 | Lactose + Cane Juice | 1:1 | 20 | <21 Mpas | Yes |
| 12 | Lactose + Molasses | 1:1 | 20 | <21 Mpas | Yes |
| 13 | Cane Juice control | N/A | 20 | <21 Mpas | N/A |
| 14 | Molasses control | N/A | 20 | <21 Mpas | N/A |

*These solutions were fully dissolved but formed suspensions after overnight refrigeration.

The dextrin used was digestive resistant dextrin derivative.

TABLE 2
spray drying of solutions of Table 1
Each solution was filtered before spray drying.
The preferred method was stocking filtration.

| | Gun | Top | Bottom | Feed | |
|---|---|---|---|---|---|
| Number | Temp | Temp | Temp | pressure | Powder |
| 1 | 260 | 193 | 80 | 1.1 psi | 50% Liquid |
| 2 | 260 | 200 | 93 | 1.5 psi | 75% Liquid |
| 3 | 158 | 80 | n/a | 0.5 MPa | powder |
| 4 | 158 | 80 | 80 | 0.5 MPa | powder |
| 5 | 158 | 80 | n/a | 0.5 MPa | powder |
| 6 | 158 | 80 | n/a | 0.5 MPa | powder |
| 7 | 158 | 80 | n/a | 0.5 MPa | n/a* |
| 8 | 158 | 80 | n/a | 0.5 MPa | n/a* |
| 9 | 158 | 80 | n/a | 0.5 MPa | sticky powder |
| 10 | 158 | 80 | n/a | 0.5 MPa | sticky powder |
| 11 | 158 | 80 | n/a | 0.5 MPa | powder |
| 12 | 158 | 80 | n/a | 0.5 MPa | Powder |
| 13 | 158 | 80 | n/a | 0.5 MPa | Very sticky powder |
| 14 | 158 | 80 | n/a | 0.5 MPa | Very sticky powder |

*The calcium phosphate solutions 7 and 8 blocked the spray drier and did not produce a product.

Control solutions 13 and 14 did not include a HMWC and show that a suitable powder cannot be prepared without a HMWC additive.

Solutions 1 and 2 were spray dried using a co-current spray drier and produced liquid products. Later experiments with a co-current drier were successful but lower temperatures were used.

Solutions 3 to 14 were dried using a counter current spray drier. The drier was a pilot scale unit at Monash University. Similar results are expected if commercially available models are used. Viable powders were formed using the HMWCs inulin, hi-maize (corn starch) and lactose. The dextrin powders were too sticky for commercial use and the calcium phosphate solutions clogged the drier. However, it is expected that dextrin will be a suitable drying agent, if desiccant is added.

After a 4 week period of storage at room temperature and humidity, the inulin and hi-maize containing powders remained flowable powders. The lactose powders caked, likely due to the hygroscopicity of the lactose, but addition of a desiccant is likely to improve the shelf life of the powder.

Interestingly, there was no significant difference between the results achieved from the cane juice and molasses solutions. Two minor differences were that Hi-Maize with molasses formed a stickier (but still acceptable) powder than Hi-Maize with cane sugar and inulin with molasses resulted in a greater yield of non-sticky powder than inulin with cane sugar.

### Example 2

### Analysis of Polyphenol Content in Amorphous Sugar, Cane Juice or Molasses

40 g of sample was accurately weighed into a 100 ml volumetric flask. Approximately 40 ml of distilled water was added and the flask agitated until the sample was fully dissolved after which the solution was made up to final volume with distilled water. The polyphenol analysis was based on the Folin-Ciocalteu method. In brief, a 50 μL aliquot of appropriately diluted raw sugar solution was added to a test tube followed by 650 μL of distilled water. A 50 μL aliquot of Folin-Ciocalteu reagent was added to the mixture and shaken. After 5 minutes, 500 μL of 7% Na₂CO₃ solution was added with mixing. The absorbance at 750 nm was recorded after 90 minutes at room temperature. A standard curve was constructed using standard solutions of catechin (0-250 mg/L). Sample results were expressed as milligrams of catechin equivalent (CE) per 100 g raw sample. The absorbance of each sample sugar was determined and the quantity of polyphenols in that sugar determined from the standard curve.

An alternative method for analysis of the polyphenol content is to measure the amount of tricin in a sample using near-infrared spectroscopy (NIR). In these circumstances, (where the polyphenols are sourced from sugar cane) the amount of tricin is proportional to the total polyphenols. Further information on this method is available in Australian Provisional Patent Application No 2016902957 filed on 27 Jul. 2016 with the title “Process for sugar production”.

Sucrose sugars with 20 to 45 mg CE polyphenols/100 g carbohydrates and 0 to 0.5 g/100 g reducing sugars are known to have low GI (see international patent application no. PCT/AU2017/050782). Sucrose sugars with 46 to 100 mg CE polyphenols/100 g carbohydrates and 0 to 1.5% w/w reducing sugars (with not more than 0.5% w/w fructose and 1% w/w glucose) are also known to be low GI (see Singaporean patent application no. SG 10201807121Q).

### Example 3

### Analysis of the Reducing Sugar Content in Amorphous Sugar, Cane Juice or Molasses

There are several qualitative tests that can be used to determine reducing sugar content in a sample. Copper (II) ions in either aqueous sodium citrate or in aqueous sodium tartrate can be reacted with the sample. The reducing sugars convert the copper(II) to copper(I), which forms a copper(I) oxide precipitate that can be quantified.

An alternative is to react 3,5-dinitrosalicylic acid with the sample. The reducing sugars will react with this reagent to form 3-amino-5-nitrosalicylic acid. The quantity of 3-amino-5-nitrosalicylic acid can be measured with spectrophotometry and the results used to quantify the amount of reducing sugar present in the sample.

### Example 4

### Determining the Amount of Solids Dissolved in Cane Juice or Molasses

A volume of the cane juice or molasses is filtered into a flask via a stocking. A petri dish is weighed and several drops of cane juice are placed on the petri dish and quickly re-weighed to avoid any moisture loss to the surrounding air. The petri dish is then left in an oven containing desiccant pellets at 70° C. overnight and weighed the following day. The sample is re-weighed and left in the oven until a consistent mass is observed. This mass is devoid of moisture and is the total amount of solid from the drops of cane juice.

After being weighed, the mass can be calculated against the initial mass to find the mass fraction of total solids in the cane juice for further dilution.

### Example 5

### Ratios of Drying Agent to Total Solids Tested

Once the total solids are tested, the drying agent (either hi-maize (HM), lecithin, whey protein isolate (WPI) or a combination thereof) is added in the specified mass ratio. The various solutions are then diluted to the final total solids percentage for the feed to be dried, and mixed thoroughly using a magnetic stirrer. The ratios and TS values of the tested samples are in Table 4.

TABLE 4
Spray dried cane juice prepared using the counter current
spray drier as used in Example 1 with varied amounts of
total solids (TS), ratios of cane juice (CJ), Whey Protein
Isolate (WPI) and Hi-Maize (HM) and inlet air temperature.

| Test | Total Solids | CJ:WPI:HM | Inlet Air |
|---|---|---|---|
| No. | (TS) % | (TS) | Temperature (° C.) |
| 1 | 10 | 70:30:0 | 160 |
| 2 | 10 | 80:20:0 | 160 |
| 3 | 10 | 90:10:0 | 160 |
| 4 | 10 | 95:5:0 | 160 |
| 5 | 10 | 98:2:0 | 160 |
| 6 | 10 | 99:1:0 | 160 |
| 7 | 10 | 99.5:0.5:0 | 160 |
| 8 | 10 | 50:0:50 | 160 |
| 9 | 10 | 60:0:40 | 160 |
| 10 | 10 | 70:0:30 | 160 |
| 11 | 10 | 80:0:20 | 160 |
| 12 | 10 | 90:0:10 | 160 |
| 13 | 10 | 60:30:10 | 160 |
| 14 | 10 | 60:20:20 | 160 |
| 15 | 10 | 60:30:10 | 160 |
| 16 | 10 | 60:35:5 | 160 |
| 17 | 10 | 60:38:2 | 160 |
| 18 | 10 | 60:39:1 | 160 |
| 19 | 10 | 60:39.5:0.5 | 160 |

Results—Yield

Bulk Density

Two bulk density values were determined for the powder that was produced; free poured powder bulk density, and tapped density.

In order to determine the free poured density, a 20 g mass of powder was poured into a graduated measuring cylinder and the volume occupied read off the cylinder markings.

Tapped bulk density for this sample will then be determined by dropping the 20 g sample in the measuring cylinder 20 times onto a rubber mat from a height of 15 cm.

Flowability

The flowability of the powder obtained from the spray drying process, is determined using the Hausner ratio, and correlated to a flow property. These flow properties are shown in Table 5 below.

TABLE 5
Details of powder flowability vs Hausner ratio

| Powder Flow Property | Hausner Ratio |
|---|---|
| Excellent | 1.00-1.11 |
| Good | 1.12-1.18 |
| Fair | 1.19-1.25 |
| Passable | 1.26-1.34 |
| Poor | 1.35-1.45 |
| Very Poor | 1.46-1.59 |
| Very Very Poor | >1.60 |

The Hausner ratio is calculated as the ratio of tapped powder density to freely poured density. This is represented in the equation below:

HR=ρT/ρF, where ρT and ρF are the tapped and free poured densities, respectively.

Moisture Content

Moisture content of the dried powders was determined by taking a 3-4 gram or 1-2 gram sample of powder, and placing this in an oven at 70° C. with a desiccant until the mass of powder remains constant. Moisture content is then determined as a percentage of the original mass of powder.

Susceptibility to Caking

Powders collected from the spray drying process were stored in zip locked bags or vacuum sealed bags, and left at either ambient and refrigerated conditions. The powder was qualitatively analysed to determine how susceptible it is to caking based on the size and number of cakes present in the powder, and also the ease of breaking up the cake (ie very easy to break up into powder again, or extremely tough and difficult to granulate).

Powder Solubility

Solubility of powder was determined by dissolving a sample of the dried product in water, and visually examining to indicate if there are any suspended solids present.

Counter Current Spray Drying

500 g of solution was spray dried in each experimental run. The feed pressure was 500 kPa. The feed flows through a nozzle type atomiser at a rate of 15 ml/min. Results are shown in Table 6 below.

TABLE 6
Spray dried CJ:WPI

| | | Inlet | | | Chamber | | | |
|---|---|---|---|---|---|---|---|---|
| | | Air | Inlet Air | Atomisation | Tempera- | | Moisture | |
| Run | | Temp | Pressure | Pressure | ture | Powder | Content | T_{g} |
| Number | CJ:WPI | (° C.) | (kPa) | (kPa) | (° C.) | produced | (%) | (° C.) |
| 1 | 70:30 | 180 | 350 | 400 | 68.0 | Yes | 8.02 | N/A |
| 2 | 80:20 | 190 | 350 | 500 | 69.8 | Yes | 9.42 | 22.81 |
| 3 | 80:20 | 200 | 350 | 500 | 72.7 | Yes | 5.03 | 26.19 |
| 4 | 80:20 | 210 | 350 | 500 | 76.0 | Yes | 6.09 | 33.49 |
| 5 | 90:10 | 200 | 400 | 500 | 72.7 | Yes | 8.82 | — |
| 6 | 90:10 | 220 | 350 | 500 | 79.5 | Yes | 6.27 | — |

Whey protein isolate was found to be a very effective additive in the spray drying of cane juice. The inlet air temperature was increased in 10° C. increments twice, whilst retaining the same feed solution conditions and it was found that the driest powder that displayed high flowability and minimal caking following storage was produced at an inlet air temperature of 200° C., with a moisture content of 5.03%.

It was initially thought that powder produced utilising higher temperatures would be drier than those produced at lower temperatures, however it was found that there existed an optimum temperature that would yield powders with minimal water remaining, and operating at temperatures higher or lower than this point would increase the residual moisture. FIG. 2 depicts moisture content versus temperature of the drying chamber.

Without being bound by theory, it is thought that the increase in air temperature increases the rate of evaporation from the droplet to the air resulting in lower moisture content until the evaporation occurs too rapidly and a crust is formed on the surface of the particle, which slows further evaporation from the particle, resulting in an increase. Using a similar inlet air temperature but only 10% drying agent increased the water content. When the inlet air temperature was increased to 220° C., the moisture content of the powder lowered back to 6.27%. The best sample with 20% WPI remained completely free flowing with no caking upon storage (row 3, Table 6) and is therefore the best of the sugars prepared.

The optimum ratio of cane juice to WPI was found to be 80:20 CJ:WPI at a total solids concentration of 20% w/w. Drying chamber temperature was found to have a significant influence on the stability of the powders formed, ultimately as a result of residual moisture content in the powder. An inlet air temperature of 200° C. corresponding to an average drying chamber temperature of 72.7° C. was found to give the lowest moisture content of the 80:20 powder at 5.03%. This yielded a free flowing, stable powder that did not exhibit caking.

Results of spray drying compositions comprising lecithin are shown in Table 7 below.

TABLE 7
Spray dried CJ:WPI:L

| | | Inlet | | | Chamber | | | |
|---|---|---|---|---|---|---|---|---|
| | | Air | Inlet Air | Atomisation | Tempera- | | Moisture | |
| Run | | Temp | Pressure | Pressure | ture | Powder | Content | T_{g} |
| Number | CJ:WPI:L | (° C.) | (kPa) | (kPa) | (° C.) | produced | (%) | (° C.) |
| 10 | 80:15:5 | 200 | 350 | 500 | 72.7 | Yes | 6.85 | — |
| 11 | 80:10:10 | 200 | 350 | 500 | 72.7 | Yes | 5.33 | 52.76 |
| 12 | 80:5:15 | 200 | 350 | 500 | 72.7 | Yes | 4.14 | 35.2 |
| 13 | 90:7.5:2.5 | 200 | 350 | 500 | 72.7 | Yes | 5.62 | — |
| 14 | 90:2.5:7.5 | 200 | 350 | 500 | 72.7 | Yes | 4.48 | — |
| 15 | 95:1.25:3.75 | 200 | 350 | 500 | 72.7 | Yes | 5.74 | — |

Items 11 and 12 were also shown to remain free flowing and not cake upon storage.

The addition of lecithin improved the moisture content when compared to the use of WPI alone. As expected, flowability and storage stability were also improved. The powders that were dried using a ratio of 3:1 lecithin to WPI in the drying agent had moisture contents as low as 4.14%.

By adding lecithin, it was possible to produce powders with as little as 95:5 (CJ:Total Drying Agent) that did not cake upon storage.

The optimum ratio of WPI:Lecithin was determined to be 1:3, and using a ratio of 80:5:15 CJ:WPI:L the moisture content of 4.14% was achieved. Furthermore the addition of Lecithin eliminated wall deposition of powder in the spray dryer.

### Example 7

### Effect of Inlet Temperature and Protein Ratio

Food grade sucrose (CSR) and Whey protein (Bulk Nutrients) were used to prepare the Sucrose-protein model solutions of Table 8 below. Distilled water at room temperature was used to dissolve sucrose and whey protein in a 2 L glass beaker by a magnetic stirrer. The same spray drier was used as for Examples 1 and 5.

TABLE 8
testing refined sugar model solutions

| | Inlet air | Solid in | | | | |
|---|---|---|---|---|---|---|
| | tempera- | total | Sucrose: | | Moisture | |
| | ture | solution | protein | Yield | content | |
| Trial | (° C.) | (wt %) | ratio | (wt %) | (%) | Stability |
| 1 | 160 | 10 | 90:10 | 4.4 | 3 | Free flowing |
| 2 | 160 | 20 | 90:10 | 11 | 9 | Free flowing |
| 3 | 160 | 40 | 90:10 | 29.2 | 14 | Sticky and |
| | | | | | | caking |
| 4 | 180 | 20 | 90:10 | 17 | 10 | Free flowing |
| 5 | 180 | 40 | 90:10 | 20.8 | 10 | Free flowing |
| 6 | 180 | 20 | 95:5 | 8.5 | 7 | Sticky, free |
| | | | | | | flowing |
| 7 | 180 | 40 | 95:5 | 9.7 | 14 | Sticky and |
| | | | | | | caking |

10% WPI of the total solids (WPI plus sucrose) was required for a non-sticky product, 5% being insufficient drying agent. Suitable powders had less than 14% moisture.

10, 20 and 40% solids in solution with a 90:10 sucrose to protein ratio resulted in free flowing powder using inlet air at 160° C. (10%) or 160° C. and 180° C. (20 and 40%).

The best yield was at 160° C. with 40% solids in solution at 90:10 sugars to WPI. However, the resulting powder was sticky possibly because the temperature was too low for the quantity of solids. The % total solids suitable varies between spray driers and the skilled person is able to optimise the % total solids. Increasing the temperature to 180° C. resolved the stickiness and retained a good yield. However, lower moisture content was considered more likely to result in a long shelf life.

Therefore, the preliminary study indicated that 160° C. to 180° C. with 90:10 sucrose:WPI were settings worth optimising for the low GI sugar of the invention.

### Example 8

### Low GI Sugars Prepared with Co-Current Spray Drier

Materials

Sugar cane juice.

Non-flavoured WPI from Bulk Nutrients

Feed solution mixture for spray drying was 40% w/w. The co-current spray dryer used had capacity to atomize high % feed solutions. A 90:10% cane juice to WPI solids solution was prepared: 1440 g sugar cane juice and 160 g WPI (20% w/w in solid base) were mixed with 2400 g Milli-Q filtered water and stirred well.

Equipment

Spray dryer in the experiments is fabricated by KODI Machinery co. LTD. Model is LPG-5. Scanning Electron Microscope (SEM) is used to analyse the particle morphology. SEM model is PhenomXL Benchtop. The test sample is coated by Sample Coater (Quorum SC7620 Sputter coaster) prior to analysis.

Method

The spray drier was set to inlet temperature 170° C. and outlet 62° C. and the feed stock spray dried.

Results

A free flowing powder is produced with 1% moisture and over 70% yield. The product does not cake and has good stability.

80:20 and 70:30 CJ:WPI % solids sugars were also prepared.

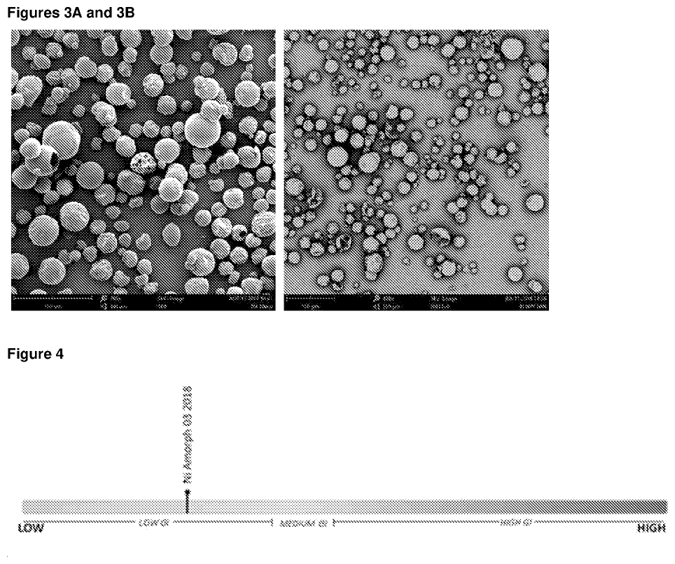

SEM images of the 80:20 and 70:30 CJ:WPI % solids sugars are in FIGS. 3 and 4 respectively. There is some porosity in the 80:20 sugar. The 70:30 sugar shows more “chipped” or “damaged” particles. The porous and chipped particle sugars remain of commercial interest.

### Example 9

### GI Testing

Part A—GI Testing of 90:10 CJ:WPI Sugar from Example 8

FIG. 4 graphs the results of an in vitro Glycemic Index Speed Test (GIST) on the 90:10 CJ:WPI sugar from Example 8. The testing involved in vitro digestion of the sugar and analysis using Bruker BBFO 400 MHz NMR Spectroscopy. The testing was conducted by the Singapore Polytechnic Food Innovation & Resource Centre, who have demonstrated a strong correlation between the results of their in vitro method and traditional in vivo GI testing. The 90:10 cane juice to whey protein isolate % solids amorphous sugar is low glycaemic.

As the 90:10 sugar is low GI, the skilled person would expect the higher protein 80:20 and 70:30 sugars to also be low GI. The skilled person would also expect similar results for amorphous sugars with different drying agents, such as fibre, so long as the drying agent has no GI (like protein) or is low GI. Insoluble fibres have little effect on GI so the GI of the amorphous sugar should remain low when an insoluble fibre is the drying agent. Soluble fibres lower the glycaemic index so amorphous sugars having a soluble fibre drying agent will have even lower GI than the tested sugars with a protein drying agent. High intensity sweeteners like stevia or monk fruit sweeteners have a GI of zero. Therefore, amorphous sugars with high intensity sweeteners as a drying agent will also remain low GI.

The polyphenol content of the 90:10 CJ:WPI % solids amorphous sugar was tested for polyphenol content at the Singapore Polytechnic Food Innovation & Resource Centre using the Folin-Ciocalteu assay (UV detection at 760 nm) using an Agilent Cary 60 UV-Vis Spectrophotometer. The sugar has 446.80 mg CE polyphenols/100 g carbohydrates.

Part B—Preparation of Sugar with Very Low GI

The effect of polyphenol content on the GI of sugar was studied. Traditional white sugar ie essentially sucrose was used as a control. Sugars with varied quantities of polyphenols were prepared by adding various amounts of polyphenol content to traditional white sugar.

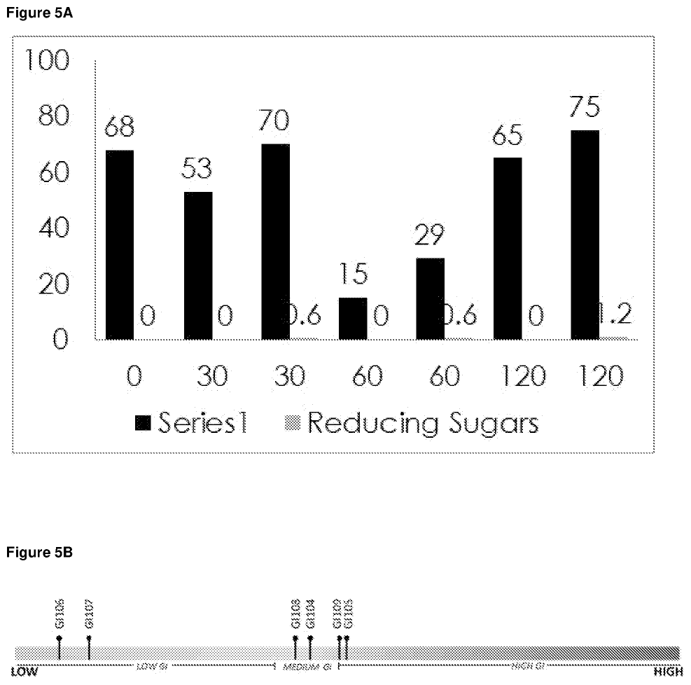

Table 9 shows the results of testing of an in vitro Glycemic Index Speed Test (GIST) on the sugars prepared. The method involved in vitro digestion and analysis using Bruker BBFO 400 MHz NMR Spectroscopy. The testing was conducted by the Singapore Polytechnic Food Innovation & Resource Centre, who have demonstrated a strong correlation between the results of their in vitro method and traditional in vivo GI testing. The results of the GIST testing is also graphed in FIG. 5A.

TABLE 9
Sugar polyphenol content v GI

| Sample | Polyphenol content | GI number | GI |
|---|---|---|---|
| 1 | 0 mg CE/100 g | About 68 | Medium |
| 2 | 30 mg CE/100 g | <55 (about 53) | Low |
| 3 | 60 mg CE/100 g | <20 (about 15) | Very Low |
| 4 | 120 mg CE/100 g | <68 (about 65) | Medium |

While the GI of fructose is 19, the GI of glucose is 100 out of 100. We therefore expect that the as glucose increases in less refined sugars the glycemic response also concurrently increases.

A second set of sugars were prepared in which reducing sugars (1:1 glucose to fructose) were added to some of the white refined sugar plus polyphenol sugars. The GI of these sugars was also tested using the GIST method and the results are in Table 10.

TABLE 10
Effect of polyphenol and reducing sugar content on GI

| Sample # | Name of Material/Sample | Sample Code | GI Banding |
|---|---|---|---|
| 1 | Sugar + 30mg/100 g PP + | GI103 | Low |
| | <0.16% RS | | |
| 2 | Sugar + 30mg/100 g PP + | GI104 | Medium |
| | 0.3% RS | | |
| 3 | Sugar + 30mg/100 g PP + | GI105 | Medium/High |
| | 0.6% RS | | (about 70) |
| 4 | Sugar + 60mg/100 g PP + | GI106 | Very low |
| | 0% RS | | (about 15) |
| 5 | Sugar + 60mg/100 g PP + | GI107 | Low |
| | 0.6% RS | | (about 29) |
| 6 | Sugar + 120mg/100 g PP + | GI108 | Med |
| | 0% RS | | (about 65) |
| 7 | Sugar + 120mg/100 g PP + | GI109 | High |
| | 1.2% RS | | (about 75) |

*PP = polyphenols;
RS = reducing sugars (1:1 glucose:fructose)

The GI of several samples from Table 10 are graphed in FIG. 5B.

While this testing used crystalline sugar, the results are expected to apply to amorphous sugars with drying agents having no GI (eg protein, insoluble fibre or a high intensity sweetener). Other drying agents (such as soluble fibre may lower the GI further but are not expected to increase the GI).

Previous low GI sugars had a glucose based glycaemic index of about 50. The ability to prepare a very low glycaemic sugar achieving a GI of about 15, which is significantly less than half of the GI of previous low glycaemic sucrose sugars, is very surprising. In addition, it is surprising that the very low glycaemic sugar is palatable.

### Example 10

### Taste Profile for Sugars from Example 8

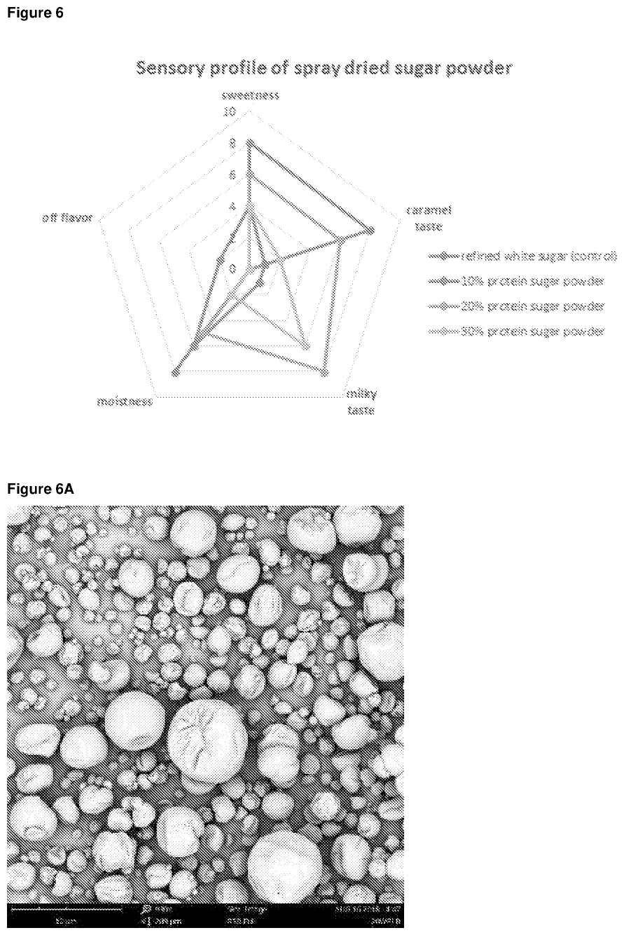

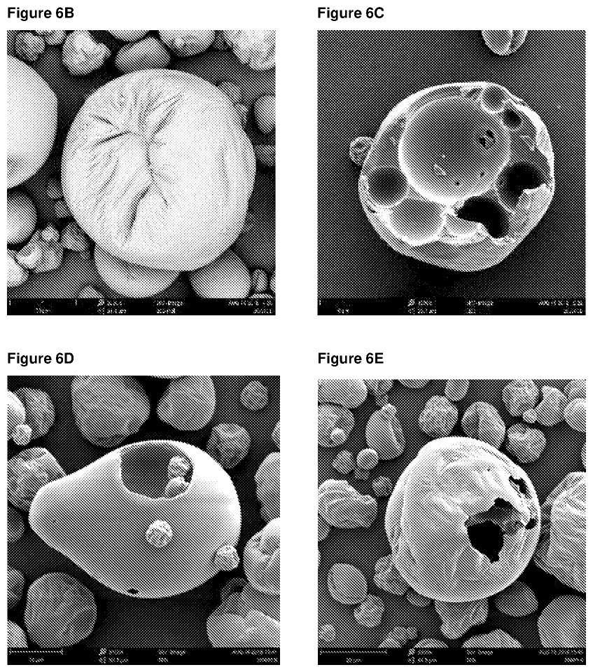

The 90:10, 80:20 and 70:30 sugars from Example 8 were taste tested by two qualified sensory analysts and two project researchers. The sensory profile is in FIG. 6.

The 90:10 and 80:20 sugars are sweeter than refined white sugar, while the 70:30 is equivalently sweet. The 90:10 and 80:20 sugars have a caramel taste. Without being bound by theory, this taste is thought to be associated with the cane juice. The 80:20 and 70:30 sugars have a milky taste. Without being bound by theory, the milky taste is thought to be associated with the WPI.

The 80:20 sugar had a good balance of sweet, milky and caramel tastes. The porosity of the particles did not cause a taste issue.

This testing demonstrates how low GI sugars can be prepared with different flavours for different applications.

### Example 11

### Aerated Amorphous Sugar

Materials:

1) sugar cane juice.

2) Whey Protein Isolate from BULK NUTRIENTS

3) feed solution mixture (50% w/w):
1600 g sugar cane juice (40% w/w of solution)
400 g WPI (20% w/w in solid base) (10% w/w of solution)
2000 g Milli-Q water (50% w/w)

Equipment:

1) Spray dryer: KODI Machinery co. LTD, Model: LPG-5

2) Scanning Electron Microscope (SEM): Phenom Benchtop SEM: Phenom XL

3) Sample coater: Quorum SC7620 Sputter coater.

Test Procedure:

1) Combine the feed solution ingredients.

2) Aerate the feed solution before atomization (by hand using a stirring rod) and create creamy/stable bubble. Stirring was consistent during drying.

2) Spray the solution into the dryer (Inlet 170° C.±1° C., outlet 62° C.±2° C., nozzle size 50 mm) to prepare the aerated amorphous sugar particles.

3) Collect powder from spray dryer. Coat the sample by Quorum SC7620 Sputter coater to prepare them for SEM analysis.

4) SEM analysis.

Results and Discussions

Aerated amorphous sugar particles were successful prepared. SEM images of the sugar powder are shown in FIG. 6A-E. The particle size is variable from less than 10 μM to about 60 μM. The aeration/porous nature of the particles is visible in the images of particles that are chipped or incompletely encased.

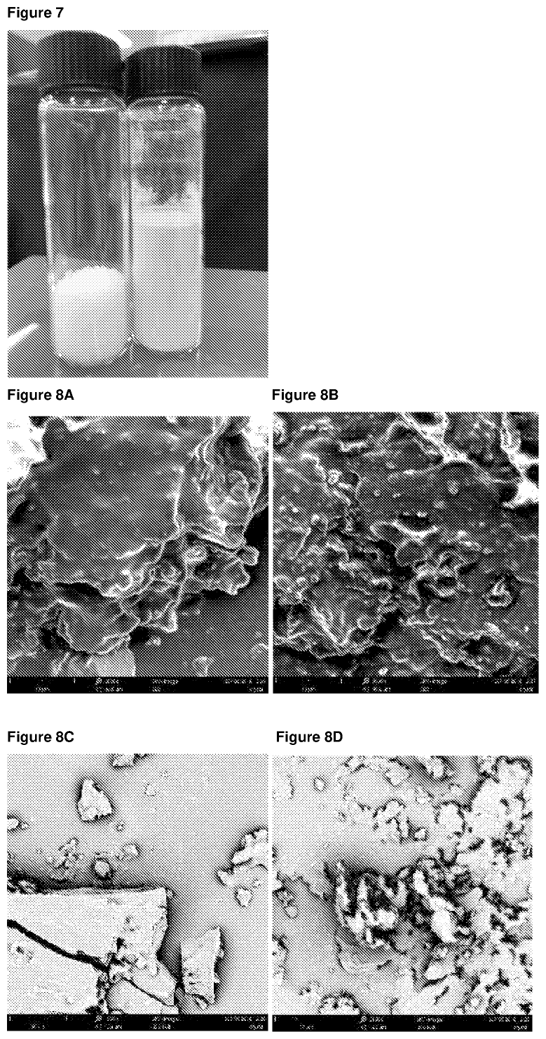

The aeration results in a low bulk density for the particles. FIG. 7 shows an image of 3 g of white crystal sugar and 3 g of the aerated amorphous sugar prepared according to this example. The bulk density of the white sugar is about 0.88 g/cm³. The bulk density of the aerated amorphous sugar is about 0.47 g/cm³.

### Example 12

### Sugar Reduction Potential of the Amorphous Sugar

The composition of the sugar prepared in Example 8 was analysed using Near Infrared technology by FeedTest Laboratory in Australia. The results of the analysis are in Table 11 below.

TABLE 11
composition of the 20% WPI:CJ amorphous sugar

| TEST | Result |
|---|---|
| Crude Protein (TP/026) | |
| Protein (N × 6.25) | 23.5 |
| (% of dry matter) | |
| Fat by Acid | |
| Hydrolysis (TP/050) | |
| Fat (dmb) | 1.1 |
| (% of dry matter) | |
| Ash (TP/024) | |
| Ash (dmb) | 7.6 |
| (% of dry matter) | |
| Crude Fibre (TP/098) | |
| Crude Fibre (dmb) | 1.1 |
| (% of dry matter) | |
| NFE (TP/FT/008) | |
| NFE (%) | 62.5 |
| Metabolisable Energy (Atwater) | |
| (TP/FT/008) ∧ | |
| ATWATER_ENERGY | 346.1 |
| (kcal/100 g dry matter) | |
| Dry Matter (FT/002) ∧ | |
| Dry Matter (%) | 98.3 |
| Moisture (%) | 1.7 |
| Starch (TP/037) ∧ | |
| Total Starch (% of dry matter) | 0.9 |
| Sugar Profile (TP/036) | |
| Total Free Sugars (%) | 63 |

Crude fibre is the insoluble carbohydrate and NFE (Nitrogen free extract) is the soluble carbohydrate.

This amorphous sugar has 63% free sugars compared to 100% free sugars for refined white sugar, yet the sweetness of the sugar is comparable (see Example 11 and FIG. 6). This is a 37% reduction in sugar if the amorphous sugar is substituted for white refined sugar in a 1:1 ratio (by weight). However, based on the increased sweetness a substitution of 0.85:1 could be achieved. This would result in a 43% reduction in free sugar. The results for a non-aerated version of the sugar are expected to be identical as this comparison is based on weight not density/volume.

Where the sugar source for the amorphous sugar of the invention is sugar cane juice (or something with equivalent composition), the reduction in free sugar is expected to be equivalent independent of the drying agent used (so long as the drying agent does not include free sugar).

White refined sugar is 1,700 kJ/100 g. This amorphous sugar is about 346 kcal/100 g, which is about 1448 kJ/100 g. Therefore, the amorphous sugar contains about 85% of the total energy/total calories of white refined sugar. In other words, the total energy/total calories by weight of the amorphous sugar is reduced by 15% when compared to an equivalent weight of white refined sugar. These calculations are based on an aerated sugar and protein blend. The protein included has calories. Non-digestible/digestive resistant foods will have lower to no calories. A sugar with a non-digestible/digestive resistant ingredient instead of a protein will have increased calorie reduction.

Again, the results for a non-aerated version of the sugar are expected to be identical as this comparison is based on weight not density/volume.

The skilled person will understand that the reduction in total energy will vary depending on the nature and amount of the drying agent used. For example, if the drying agent is a fibre, a larger reduction in total energy is expected than where the drying agent is protein. A larger reduction in total energy is expected where a greater amount of drying agent is used, for example, 30% by solid weight.

The nutritional information for the composition of the sugar prepared in Example 8 is in Table 12 below. The % Daily Value (DV) in the table tells you how much a nutrient in a serving of food contributes to a daily diet. 2,000 calories a day is used for general nutrition advice.

TABLE 12
nutritional details of a serving size

| Serving size | 100 g |
|---|---|
| Calories | 350 |
| Content in % Daily Value | |
| Total fat 1 g | 1% |
| Saturated fat 0 g | 0% |
| Trans fat 0 g | 0% |
| Cholesterol 0 mg | 0% |
| Sodium 170 mg | 7% |
| Total Carbohydrate 63 g | 23% |
| Dietary Fiber 1 g | 4% |
| Total sugars 63 g | |
| Includes 0 g | 0% |
| added sugars | |
| Protein 24 g | 48% |
| Vitamin D 0 mcg | 0% |
| Calcium 1200 mg | 90% |
| Iron 29 mg | 160% |
| Potassium 170 mg | 35% |
| Magnesium | 70% |
| Zinc | 30% |
| Copper | 60% |
| Manganese | 350% |

This sugar has significantly more mineral content than traditional white crystal sugar.

Traditional white crystalline sugar is about 400 calories per 100 g serve. This 20% solids w/w whey protein isolate and 80% w/w solids sugar cane juice amorphous sugar has 87.5% of the calorie content of an equivalent mass of traditional crystalline white sugar. This is a reduction in calories of 12.5%. The protein in this sugar has calories, if a non-digestible carbohydrate drying agent was used, the calories present would be reduced and the calorie reduction larger. The results will be the same whether or not the sugar is aerated as density is not relevant to this measure.

As mentioned previously, as this amorphous sugar is sweeter than traditional sugar, it is thought that a substitution of 0.85:1 could be achieved. This would result in an about 25.6% reduction in calories by weight.

### Example 13

### Preparation of Chocolate Using Aerated Amorphous Sugar

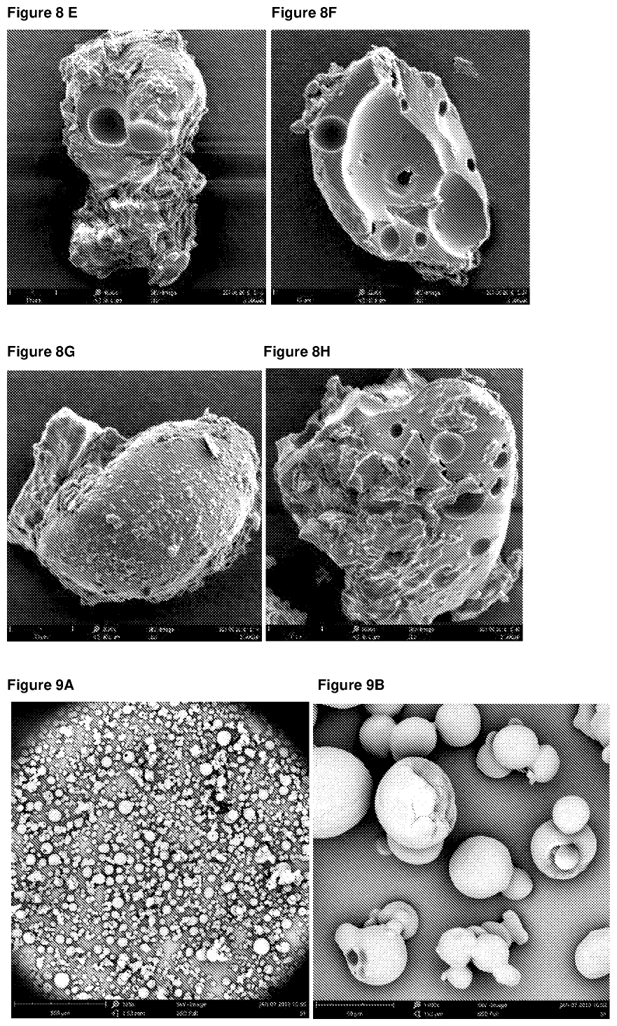

30 g of Lindt 70% dark chocolate was melted and combined with 30 g white crystalline sugar as a control. 30 g of Lindt 70% dark chocolate was melted on a water bath, mixed with 15 g aerated amorphous sugar prepared according to Example 8 and allowed to set. SEM images were taken using the SEM process described in Example 8 and are depicted in FIG. 8—A to D showing the chocolate with sugar crystals; and E to H showing the chocolate with the aerated amorphous sugar.

FIGS. 8A-D indicate solid chocolate with tactile sugar crystals. FIGS. 8E-H indicate the chocolate is coated onto the aerated amorphous sugar particles. The chocolate coated amorphous particles are less than 25 μm and no bigger particles were detected.

Both Samples Were Taste Tested

Solid chocolate with tactile sugar crystals: The first taste is bitter from cocoa. The sweetness comes quite late in aftertaste. Overall taste is less sweet than the chocolate coated aerated amorphous sugar particles despite the high sugar content.

Chocolate coated aerated amorphous sugar particles: First taste is sweet. The texture is creamy and full of aroma. The aftertaste is still sweet. The overall taste is almost double the sweetness of the white sugar chocolate blend but has only 50% w/w added sugar content.

### Example 14

### Amorphous Sugars Prepared with Varied Sugar Sources

In this example, the technology developed to prepare amorphous sugars was applied to prepare amorphous alternative sweeteners with soluble fibre, insoluble fibre or protein including vegan protein.

Materials

Recipe 1

1) Sweeteners
rice syrup—Pure Harvest: Organic Rice malt syrup
coconut sugar—CSR: unrefined coconut sugar
monk fruit—Morlife: Nature's Sweetener Monk Fruit
maple syrup—Woolworths: 100% pure Canadian Maple syrup

2) Whey Protein Isolate from BULK NUTRIENTS 100% WPI.

Feed Solution Mixture
360 g Sweeteners (a. Rice syrup, b. Coconut sugar, c. Monk fruit (300 grams, find the feed solution in the table below) or d. Maple syrup)
40 g WPI
600 g Milli-Q water

Recipe 2

1) Sweetener: Sugar Cane Syrup

2) Whey Protein Isolate

3) Soluble fibres (Lotus: Xanthan Gum) or insoluble fibres (KFSU: Phytocel—100% natural sugarcane flour)

Feed Solution Mixtures

3.1) Insoluble fibres
360 g Sugar Cane Syrup
36 g WPI
4 g Insoluble fibres
600 g Milli-Q water

3.2) Soluble fibres
500 g Sugar Cane Syrup
36 g WPI
4 g Insoluble fibres
400 g Milli-Q water

Recipe 3

1) Sweetener: Sugar Cane Syrup

2) Vegan Protein (Bio Technologies LLC, Sunprotein: Sunflower protein powder).

Feed Solution Mixture
500 g Sugar Cane Syrup
40 g Vegan Protein
300 g Milli-Q water

Equipment

1) Spray dryer: LPGS, KODI Machinery co. LTD.

2) Scanning Electron Microscope (SEM): Phenom Benchtop SEM: Phenom XL

3) Sample coater: Quorum SC7620 Sputter coater.

4) Vacuum Packaging Machine

Test Procedure

1) Combine and mix the feed solution ingredients to create a stable solution (as opposed to a solution with a stable bubble) before atomization.

2) Spray the solution into the dryer (Inlet 170° C.±1° C., outlet 70° C.±2° C., nozzle size 50 mm).

3) Collect powder from spray dryer. Coat the sample by Quorum SC7620 Sputter coater to prepare them for SEM analysis.

4) SEM analysis.

TABLE 13
Ingredients in the amorphous sugars of Example 14

| | Recipe | Sweetener | g | Protein | g | Fibre | g | Water (g) |
|---|---|---|---|---|---|---|---|---|
| 1 | 1 | Rice syrup | 360 | WPI | 40 | — | — | 600 |
| 2 | 1 | Coconut | 360 | WPI | 40 | — | — | 600 |
| | | sugar | | | | | | |
| 3 | 1 | Monk fruit | 300 | WPI | 40 | — | — | 600 |
| 4 | 1 | Maple syrup | 360 | WPI | 40 | — | — | 600 |
| 5 | 2 | Sugar Cane | 500 | WPI | 36 | Soluble | 4 | 400 |
| | | Syrup | | | | Xanthan | | |
| | | | | | | Gum | | |
| 6 | 2 | Sugar Cane | 360 | WPI | 36 | Insoluble | 4 | 600 |
| | | Syrup | | | | Fibre | | |
| | | | | | | Bagasse | | |
| | | | | | | (Phytocel) | | |
| 7 | 3 | Sugar Cane | 500 | Sunflower | 40 | — | — | 300 |
| | | Syrup | | protein | | | | |

Results

In each case, a free-flowing powder was formed (prior to sputter coating) and aerated amorphous sugar particles were successful prepared. The powders were aerated but less aerated than the powders prepared in Example 11, where the solution was actively aerated before spray drying using a hand stirring rod. These powders were only mixed ordinarily to achieve a homogeneous solution to spray dry rather than more vigorously mixed to achieve a stable bubble.

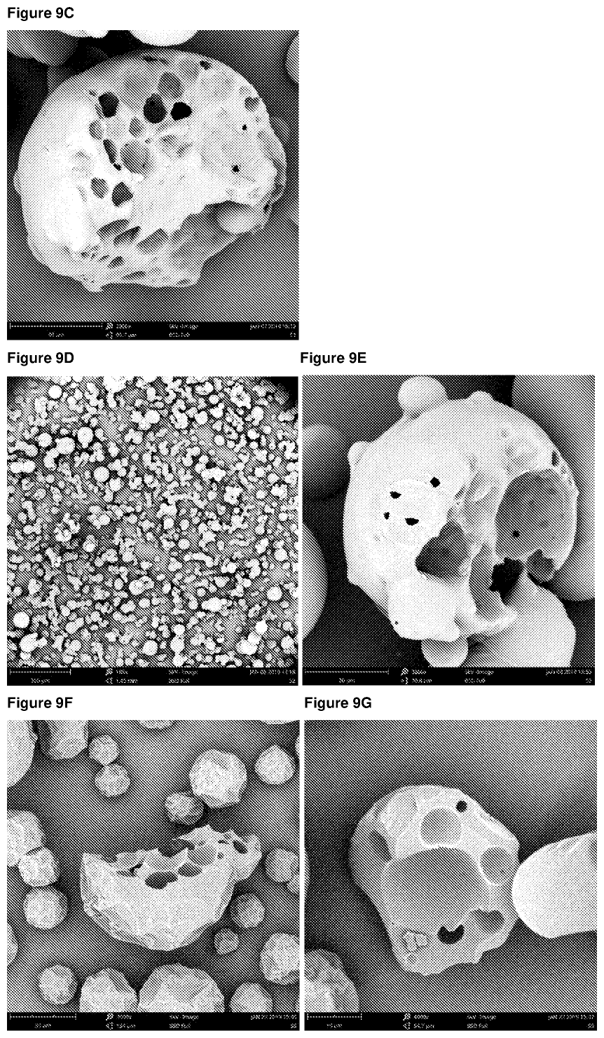

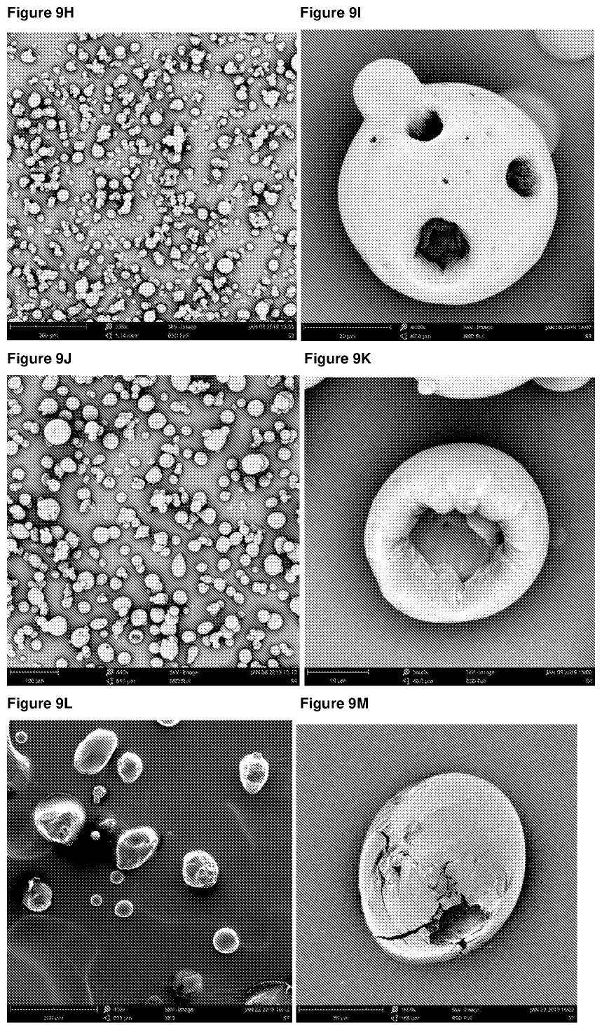

SEM images of products 1 to 4 and 6 to 7 from Table 12 are in FIG. 9A-C (rice syrup), D-E (coconut sugar), F-G (monk fruit), H-I (maple syrup), J-K (bagasse), L-M (sunflower protein). There are no images for product 5 (xanthan gum).

The particle size is variable from less than 10 μm to about 60 μm. The aeration/porous nature of the particles is visible in the images of particles that are chipped or incompletely encased.

The bulk density of the powders was determined as for the products in FIG. 7. The results are in Table 13 below.

TABLE 14
Bulk density results

| | | | | | Density |
|---|---|---|---|---|---|
| | Recipe | Sweetener | Protein | Fibre | g/cm³ |
| 1 | 1 | Rice | WPI | — | 0.36 |
| | | syrup | (10%) | | |
| 2 | 1 | Coconut | WPI | — | 0.41 |
| | | sugar | (10%) | | |
| 3 | 1 | Monk | WPI | — | 0.37 |
| | | fruit | (10%) | | |
| 4 | 1 | Maple | WPI | — | 0.41 |
| | | syrup | | | |
| 5 | 2 | Sugar | WPI | Soluble | 0.52 |
| | | Cane | (9%) | Xanthan | |
| | | Syrup | | Gum (1%) | |
| 6 | 2 | Sugar | WPI | Insoluble | 0.38 |
| | | Cane | (9%) | Fibre | |
| | | Syrup | | Bagasse | |
| | | | | (Phytocel) | |
| | | | | (1%) | |
| 7 | 3 | Sugar | Sunflower | — | 0.55 |
| | | Cane | protein | | |
| | | Syrup | (10%) | | |

The bulk density of the aerated amorphous sugar is about 0.47 g/cm³. These results are similar despite the minimal mixing before spray drying (ie the feed stock was not stirred into a creamy bubble before spray drying). The sunflower protein resulted in aeration but was not quite as effective as the whey protein isolate at 0.55% g/cm³, a 37.5% reduction compared to traditional white sugar.

The rice syrup and monk fruit results were the least dense with a nearly 60% reduction in density. As density is likely to decrease with increasing WPI, a 70% reduction in density is plausible.

### Example 15

### Baked Goods Prepared Using the Amorphous Sugar of the Invention

Both butter cookies and vanilla cupcakes were prepared using the amorphous sugar of the invention (specifically, the sugar of Example 8 prepared from 80:20% cane juice to WPI solids).

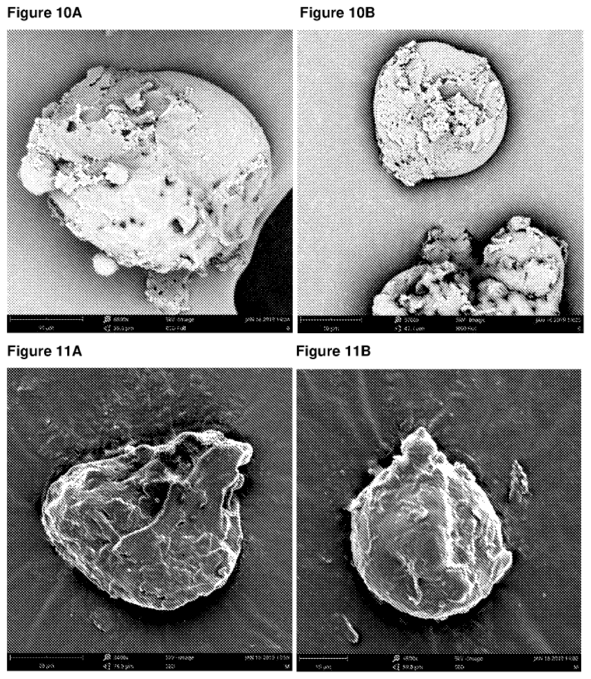

The resulting products were analysed by SEM, as shown in FIGS. 10 and 11. These images show that the aerated sugar particles remained intact in both the muffin and cookie product and had not lost their aeration during food preparation. While the aeration is less evident due to a layer of fat coating the sugar, the particle remained aerated as it retained its pre-processing size and shape.

The cookies and cupcakes were prepared as below:

TABLE 15
Ingredients in the Butter Cookies of Example 15

| Ingredient | Quantity |
|---|---|
| Plain flour | 178 g |
| Amorphous sugar of Example | 72 g |
| 8 (prepared from 80:20% | |
| cane juice to WPI solids) | |
| Butter, softened | 113 g |
| Egg | 1 |
| Vanilla extract | 2 teaspoons |
| Baking powder | ½ tablespoon |
| Baking soda | ¼ teaspoon |
| Salt | ⅛ teaspoon |

Preparation of the Butter Cookies of Example 15

Half of the amorphous sugar of Example 8 was folded into the butter and vanilla extract. Egg was added and the mixture was mixed until combined. Sifted flour, baking powder, baking soda and salt were added and the mixture was mixed until just combined. The remaining half of the amorphous sugar of Example 8 was folded into the mixture and spoonfuls of the resulting mixture were placed on a greased baking tray and baked for 20-25 minutes at 150° C.

TABLE 16
Ingredients in the Vanilla Cupcakes of Example 15

| Ingredient | Quantity |
|---|---|
| Plain flour | 90 g |
| Amorphous sugar of Example | 75 g |
| 8 (prepared from 80:20% | |
| cane juice to WPI solids) | |
| Butter, melted | 80 g |
| Milk | 40 g |
| Egg | 1 |
| Vegetable Oil | 1 taplespoon |
| Baking powder | ¼ tablespoon |
| Vanilla extract | 1 teaspoon |

Preparation of the Vanilla Cupcakes of Example 15

Half of the amorphous sugar of Example 8 was folded into the flour. Milk, butter, eggs and vanilla extract were added to the flour and sugar mixture and the ingredients were combined. The remaining half of the amorphous sugar of Example 8 was folded into the mixture and the resulting mixture was spooned into a greased cupcake pan and baked for 20-25 minutes at 150° C.

### Example 16

### Water Activity

The water activity (or partial vapour pressure) of the sugar prepared in Example 8 (cane juice and 20% solid weight whey protein isolate) was determined to be 0.31. Water activity is measured to determine shelf-stable foods. A water activity of 0.6 or less is preferred for foods and food ingredients of this type to inhibit mould and bacterial growth.

It will be understood that the invention disclosed and defined in this specification extends to all alternative combinations of two or more of the individual features mentioned or evident from the text or drawings. All of these different combinations constitute various alternative aspects of the invention.

1-64. (canceled)

65. An amorphous sugar comprising sugar cane juice, sugar beet juice and/or molasses, and a low GI drying agent.

66. An amorphous sugar according to claim 65, wherein sugar further comprises at least about 20 mg CE polyphenols/100 g carbohydrate and is low glycaemic.

67. An amorphous sugar according to claims 66, wherein the sugar has a maximum of 1 g CE polyphenols/100 g carbohydrate.

68. An amorphous sugar according to claim 65, wherein the drying agent is selected from the group consisting of lactose, protein, low GI carbohydrates, digestive resistant carbohydrate, insoluble fibre, soluble fibre, lipids, natural intense sweeteners and/or combinations thereof.

69. An amorphous sugar according to claim 65, wherein the drying agent is (i) a digestive resistant carbohydrate selected from a soluble or insoluble fibre and a combination thereof; (ii) a protein selected from whey protein isolate, β-lactoglobulin, α-lactalbumin, serum albumin, maltodextrin, pea protein, sunflower protein, hemp protein and combinations thereof; and/or (iii) a natural intense sweetener selected from stevia, monk fruit, blackberry leaf and combinations thereof.

70. An amorphous sugar according to claim 65, wherein the drying agent is a digestive resistant carbohydrate is selected from hi-maize, fructo-oligosaccharide, inulin, bagasse, xanthan gum and digestive resistant maltodextrin and its derivatives.

71. An amorphous sugar according to claim 65, wherein the drying agent is from 5% to 40% w/w of the amorphous sugar.

72. An amorphous sugar according to claim 65, wherein the drying agent has a molecular weight of 200 g/mol to 70 kDa.

73. An amorphous sugar according to claim 65, wherein the ratio of sucrose to drying agent is 95:5 to 60:40 by solid weight.

74. An amorphous sugar according to claim 65, wherein the amorphous sugar has good or excellent powder flowability defined by a Hausner ratio of 1.18 or less.

75. An amorphous sugar according to claim 65, wherein the amorphous sugar has good or excellent powder flowability defined by a Hausner ratio of 1.18 or less following 12 months storage in ambient conditions.

76. An amorphous sugar according to claim 65, wherein the amorphous sugar further comprises particles of between 1 and 100 μm in diameter.

77. An amorphous sugar according to claim 65, wherein the amorphous sugar is sweeter and/or has a more caramel flavour than white crystalline sugar.

78. An amorphous sugar according to claim 65, wherein the amorphous sugar comprises particles including both the drying agent and the sucrose.

79. An amorphous sugar according to claim 65, wherein the amorphous sugar is aerated.

80. The amorphous sugar according to claim 65, wherein the sugar has a density of 0.3 to 0.7 g/cm³.

81. The amorphous sugar of claim 80, wherein the amorphous sugar contains about 10% or about 15% less calories than an equivalent weight of white refined sugar.

82. A food or beverage comprising or made using an amorphous sugar according to claim 65.

83. A food according to claim 82, wherein the food is chocolate, cereal or a baked good.

84. A food or beverage according to claim 82, wherein the food or beverage has reduced calories from sucrose compared to the same formulation of food or beverage prepared using traditional white sugar.
